(19) United States
(12) Patent Application Publication
(10) Pub. No.: US 2019/0307691 A1
(43) Pub. Date: Oct. 10, 2019

(54) HYDROGELS WITH LIPOSOMES FOR CONTROLLED RELEASE OF DRUGS

(71) Applicant: NORTHERN ILLINOIS RESEARCH FOUNDATION, Dekalb, IL (US)

(72) Inventors: Elizabeth GAILLARD, Dekalb, IL (US); Devi K. KARUMANCHI, Novi, MI (US)

(21) Appl. No.: 16/373,274

(22) Filed: Apr. 2, 2019

Related U.S. Application Data
(60) Provisional application No. 62/653,339, filed on Apr. 5, 2018.

(51) Int. Cl. A61K 9/127; A61K 47/69; A61K 31/575; C07K 16/22; A61K 31/198; A61P 27/02; A61K 47/32; A61K 47/36

(52) U.S. Cl. CPC A61K 9/1271; A61K 47/6903; A61K 31/575; C07K 16/22; A61K 45/06; A61P 27/02; A61K 47/32; A61K 47/36; A61K 31/198

(57) ABSTRACT

Compositions and methods are provided to control the release of small molecule and macromolecule drugs through hydrogels with dispersed stable liposomes. The mixture is formed into liposomes that are dispersed within bioabsorbable hydrogels, to release the therapeutic agents in a controlled fashion. Methods of using different compositions disclosed herein in respective therapeutic systems are also provided.

### CROSS REFERENCE TO RELATED APPLICATIONS

This application claims the benefit of priority under 35 U.S.C. § 119(e) to U.S. Provisional Patent Application No. 62/653,339, filed Apr. 5, 2018. The disclosure set forth in the referenced application is incorporated herein by reference in its entirety.

### BACKGROUND

Injectable drug formulations for sustained (controlled) drug delivery are useful in fields such as ophthalmology, oncology, contraception, substance abuse and dentistry. The formulations disclosed herein include semi solid injectable hydrogels containing liposome encapsulated drugs and preferably, free drugs. Protein drugs are an important category. These formulations include combinations of DMPC, DOPE, DSPG and cholesterol. Three combinations also include PEG chains (stealth liposomes DPPE-PEG2000) and extend drug release up to 12-16 months in vitro as well as in vivo as compared with current methods of treatment.

Hydrogels

Hydrogels are materials that absorb solvents (such as water), undergo rapid swelling without discernible dissolution, and maintain three-dimensional networks capable of reversible deformation. Hydrogels may be uncross-linked or cross-linked. Uncrosslinked hydrogels absorb water, but do not dissolve due to the presence of both hydrophobic and hydrophilic regions. Covalently cross-linked networks of hydrophilic polymers, including water soluble polymers, are traditionally denoted as hydrogels in the hydrated state. Numerous aqueous hydrogels have been used in various biomedical applications, such as, for example, soft contact lenses, wound management, and drug delivery.

The hydrogels most often cited in the literature are those made of water soluble polymers, such as polyvinyl pyrrolidone, which have been cross-linked with naturally derived biodegradable components such as those based on albumin. Bioabsorbable hydrogels are well suited for local implantation, but relatively low molecular weight molecules are rapidly released from hydrogels due to the relatively open networks of previously known hydrogels.

Sustained (Controlled) Drug Release

For treating a disease or condition, a minimum effective concentration level (called the therapeutic index) must be maintained for regular intervals at the target site. Systemically administered drugs provide 100% bioavailability and provide therapeutic effects, but also lead to side-effects because of the high drug concentration administered to the subject. Systemic administration of sustained release (controlled) drug delivery systems can accomplish both objectives with a more effective utilization of the drug and reduced side effects. Local implantation of drug delivery systems may further improve the efficiency of drug availability.

Controlled drug delivery from implantable and bioabsorbable devices have been the subject of extensive exploration, but no suitable absorbable systems are known that can deliver both water soluble and water insoluble relatively low molecular weight drugs.

The development of compositions and methods to provide controlled release delivery of relatively low molecule weight drugs presents the following challenges: the delivery matrix needs to be safe and absorbable; drug release should be controlled and sustained, while being free from “burst effects”; and the devices should be simple to fabricate to prevent denaturation of photo- and oxidation-sensitive entrapped drugs.

Liposomes

Studies on the entrapment of liposomal particles in non-absorbable hydrogels indicate the liposomes may be difficult to prepare and stabilize. Also, the non-absorbable nature of polyacrylamide hydrogels precludes implantation without subsequent retrieval. Although synthesis of polymerizable liposome vesicles also has been attempted, the complicated synthesis scheme makes entrapment of drug molecules difficult.

Liposomal drug delivery systems have been extensively considered for the intravenous administration of biologically active materials, because they were expected to freely circulate in the blood. However, liposomes are quickly cleared from the blood by uptake through the reticulo-endothelial system. Coating of liposomes with poly(ethylene glycol) has been observed to increase substantially the half-life of such active materials. The flexible and relatively hydrophilic PEG chains, however, induce a steric effect at the surface of the liposome that reduces protein adsorption and thus RES uptake.

Liposomal formulations which are very stable and have a long shelf life have been reported by the inventors. This was achieved by modifying the method of preparation and including additives to prevent damage of liposomes due to oxidation and hydrolysis. Knowledge of stable liposomes was used to encapsulate model proteins e, g, anti-VEGF drugs such as Lucentis® and Avastin®. A stable drug release of about 4.5-5 months was achieved which is around 3 times slower release than reported by others. Further, contact lenses were disclosed with muco-adhesive biopolymers which produced a release of antibiotics over 180-200 days.

Optical Disease Treatment

Over the past decade, the treatment of several retinal diseases has been revolutionized by the advent of intravitreal drug delivery. In the posterior segment of the eye, diabetic retinopathy (DR) is the leading cause of blindness in Americans of working age and the third leading cause of blindness in the US. Mismanaged diabetes leads to retinal hypoxia, which induces the Muller cells to release many vasoactive factors such as VEGF and IGF which promote angiogenesis, tissue remodeling and consequently visual impairment. Age-related macular degeneration (AMD), the major cause of blindness in the elderly population in the Western World, is a multi-factorial disease that progresses from damage of the retinal pigment epithelium. In the exudative or the wet form (10-15% cases), abnormal angiogenesis causes choroidal neovascularization under or above the pigment epithelium, inducing severe visual impairment in untreated cases.

Treatment with intravitreal injection of antibodies that inhibit vascular endothelial growth factor (VEGF) usually maintains, and often improves, vision in people suffering from neovascular age-related macular degeneration, diabetic macular edema, and macular edema from retinal vein occlusion. Without treatment, most people with these diseases develop significant central vision loss. To sustain the benefit of anti-VEGF therapy, most patients require ongoing therapy with intravitreal injections every four to eight weeks. Because of the need for ongoing therapy, the number of intravitreal injections given annually has grown exponentially.

Prolonged therapy with monthly injections of anti-VEGF medications is burdensome for patients, their families, and their treating physicians. A seven-year study showed that over the long term, the benefits of intravitreal injections are lost in many patients because of difficulties in scheduling. To alleviate the burden of frequent office visits and to provide a more effective and acceptable treatment option, a sustained release drug delivery system for anti-VEGF medication is needed.

Sustained release drug delivery devices have been successfully employed in the vitreous of the eye. A surgically implanted intravitreal implant loaded with Ganciclovir was approved by the FDA for treatment of cytomegalovirus retinitis in patients with acquired immunodeficiency syndrome. Over the past 20 years, intravitreal implants have also been introduced for sustained delivery of corticosteroids. Unfortunately, there is no approved device for the sustained-release of biologics such as anti-VEGF proteins. Biodegradable polymers like chitosan nanoparticles, polyamidoamine (PAMAM) dendrimers, poly (lactic-co-glycolic) acid (PLGA) nanoparticles are being investigated to treat inflammation. Albumin nanoparticles have been successful in sustaining the release of Ganciclovir for the treatment of cytomegalovirus retinitis. PAMAM dendrimers of carboplatin were administered into the subconjunctival space of a murine model for retinoblastoma and obtained a sustained release of drug over 22 days. However, these nanoparticles do not have a huge payload and hence cannot be used for more prolonged drug delivery.

Traditional methods used for encapsulation of proteins have been less stable or have very low encapsulation efficiency. Intravitreal injection of liposome encapsulated bevacizumab (Avastin) was well tolerated through 42 days in rabbits and the clearance of this drug from the vitreous with liposomal formulations was slower than the soluble form. However, fast release of the protein into the vitreous was not restricted.

PLGA microspheres and nanospheres have achieved a sustained release of the drug over a period of 91 days in vitro. However the disadvantage of using these systems is loss of uniformity with higher loading of the antibodies, as well as the possibility of developing acidic microenvironments due to long term degradation of the polymers.

Drug Applications

Bevacizumab is widely used as an anti-angiogenic agent, and is FDA approved for cancer treatment. It is used by ophthalmologists as an off-label intravitreal agent in the treatment of proliferative (neovascular) eye diseases, particularly for choroidal neovascular membrane (CNV) in AMD. The intravitreal half-life of Avastin is about 4.32 days, maintaining a concentration of about 10 μg/ml in the vitreous over a period of 30 days. This clinically translates to receiving an intravitreal injection monthly to prevent the growth of abnormal blood vessels. Unfortunately frequent invasive injections into the eye have also been reported to increase the incidence of infections. To circumvent these problems, the anti-VEGF drugs need to be delivered through a sustained release drug delivery system over an extended period, thereby minimizing the number of injections.

Another application of a drug delivery system is sustained release L-dopa drug delivery. L-DOPA is an amino acid precursor of neurotransmitter dopamine and is used in clinical treatment of Parkinson's disease by targeting the nigrostriatal pathway. In addition to dopamine, L-DOPA is also a precursor for melanin. The enzyme tyrosinase catalyzes the oxidation of L-DOPA to the reactive intermediate dopaquinone, eventually leading to formation of melanin oligomers. However, it has been known that L-DOPA has about 30% bio-availability owing its oxidation. Under basic conditions, L-DOPA oxidizes to dopaquinone followed by spontaneous conversion of dopaquinone to red dopachrome which eventually forms insoluble melanin granules, wherein at least 50.9% of the drug was oxidized. After 24 hr in neutral pH, 47% of the drug was oxidized to melanin. Degradation of drugs can alter their receptor binding efficiency and resulting pharmacological effects, thereby leading to altered therapeutic efficacies as well as toxicity.

Parkinson's Disease (PD) is a long-term neurodegenerative disease affecting the motor system caused due to cell death in the brain's basal ganglia and dopaminergic neurons in the nigrostriatal pathway. Patients with neovascular AMD are at 2.57 times higher risk of Parkinson disease which was attributed to the effect of indirect vascular risk factors, complement activation and inflammatory responses due to microglia activation causing neurodegeneration in retina and the brain. As for treatment of Parkinson's disease, frequent levodopa administration, controlled-release levodopa preparations, inhibitors of levodopa metabolism, and duodenal, subcutaneous and even intravenous infusions of levodopa or dopamine agonists have all been employed with this goal in mind, but all have limitations. These can be attributed to the cytotoxic effect of L-DOPA when administered in large quantities to attain the minimum effective concentration for controlling the disease.

Oral administration of L-DOPA is a common treatment for Parkinson's disease, where the molecule behaves as a pro-drug for dopamine, which is unable to cross the blood-brain barrier. L-DOPA has relatively poor oral bioavailability of 30%, with an estimated less than 1% of the unmetabolized drug reaching the brain, in the absence of decarboxylase inhibitors. Even with decarboxylase inhibitors, side effects of oral L-DOPA supplements are evident in the fluctuations of plasma concentrations coupled to motor fluctuations.

Naltrexone® is an opioid receptor antagonist used primarily in the management of alcohol dependence and opioid addiction. Naltrexone hydrochloride is sold under the brand names Revia® and Depade®. In pill form, Naltrexone is usually prescribed to be taken once a day. Studies have looked at the use of Naltrexone over a 12-week period to help people who have stopped drinking to reduce the craving for alcohol during the early days of abstinence when the risk of a relapse is the greatest, although it may be used for longer in clinical practice. Because Naltrexone blocks the effects of opioids, it is also sometimes prescribed for extended periods for people trying to manage drug dependence. Increased blood concentrations of naltrexone have several side-effects such as upset stomach, nervousness, anxiety, or muscle and joint pain. Naltrexone causes more severe side effects including confusion, drowsiness, hallucinations, vomiting, stomach pain, skin rash, diarrhea, or blurred vision. Large doses of Naltrexone can cause liver failure.

Drugs by themselves are very expensive, and the physicochemical properties of drugs make it hard to deliver it to the target site, increasing the overall frequency of administrations. There is a need for an economic drug delivery system that is designed for sustained release of the drug in the patient, thereby providing the advantage of patient compliance and decreased overall cost of healthcare.

### SUMMARY

A drug product formulation includes liposome encapsulated drugs, for example protein drugs, in an injectable viscous hydrogel and may include free drug and excipients that stabilize the drug.

A cocktail of liposomes with varying release profiles entrapped in an injectable semisolid porous hydrogel, is injected into a hollow implantable matrix device for controlled release of a substance. The liposome cocktail may include a mixture of equal parts of (A) conventional liposomes with DMPC:DOPE:DSPG:Cholesterol in ratios of 60:10:0:30, 65:5:5:25 and 60:5:5:30 and (B) stealth liposomes DMPC:DSPE-PEG2000:DSPG:Cholesterol in ratios of 60:10:0:30, 65:5:5:25 and 60:5:5:30. Each composition (different phospholipid cholesterol ratios) has a different encapsulation efficiency and release profile. Each formulation has equal parts of 60:10:0:30, 65:5:5:25 and 60:5:5:30 and stealth liposomes. Each has a different release profile and can be used to encapsulate different therapeutics; wherein only 25% of a standard dose is administered through the formulation to achieve a therapeutic dose at regular intervals of time. 25% of a standard dose encapsulated within the liposome-hydrogel complex is administered through the formulation to achieve a therapeutic dose at regular intervals of time. Suitable therapeutics include antibodies, antagonists or agonists. Antibodies are encapsulated within a cocktail of liposomal formulations to prolong the time of drug release into the eye, thereby, decreasing the frequency as well as the cost factor for these treatments. The compositions are suitable to treat retinal diseases, glaucoma, and similar conditions.

Other diseases and conditions are also disclosed as suitable targets. The hydrogel may be used as a coating on wound dressings for wound healing, wherein two or more wound healing agents are encapsulated within the hydrogel and nanoparticles. The wound may be a slow healing wound, a diabetic foot ulcer, a pressure ulcer, a neural injury, traumatic brain injury, a dental injury, a cardiac injury, an ischemic brain injury, a spinal cord injury, a periodontal injury, a tendon or ligament injury, a venous leg ulcer, an ischemic ulcer, a bed sore, and a wound with a biofilm or a corneal ulcer. The hydrogel can be applied topically as a coating or a liquid bandage.

Compositions and methods are disclosed that enhance the targetability of microencapsulated drug carriers which may be readily prepared and administered, but are still highly specific in finding the target tissue and in delivering the drug efficiently. Such “smart or stealth nanoparticles” “stealth liposomes” achieve improved targeting by having lower circulation velocity, slower clearance from circulation, and by possessing selective adhesion to selected cellular targets.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

FIG. 1(A) and 1(B): FIG. 1(A) Screening the formulations based on concentration of phospholipids and cholesterol, FIG. 1(B) method of preparation.

FIGS. 2(A) and 2(B): SAXS data for determining the encapsulation of drugs based on electron density profiles.

FIG. 3: TEM images of conventional liposomes without any modifications.

FIG. 4: Drug release profiles from different compositions of conventional liposomes.

FIG. 5: TEM images of stealth liposomes.

FIG. 6: Drug release profiles from different compositions of stealth liposomes.

FIG. 7: Implant with multiple coatings comprising free drug and liposome encapsulated drug in multiple layers.

FIG. 8: Release profiles of drug from hydrogels with varying concentrations of hydrogels.

FIG. 9: Release profiles of drug using different reservoirs filled with hydrogels and controls.

FIG. 10: Release profiles of drug using hydrogel with embedded cocktail of drug encapsulated liposomes.

FIG. 11: TEM of liposome cocktail embedded in the hydrogel.

FIG. 12: Individual drug release profiles from the compositions in the cocktail of liposomes.

FIG. 13(A)-13(B): 13(A) shows particle size and 13(B) encapsulation efficiency of the liposome cocktail.

FIG. 14: Comparison of drug concentration measured in vitro using spectrophotometry with ELISA.

FIG. 15: Cell studies using avastin.

FIG. 16: Cell studies using avastin loaded liposomes.

FIG. 17: Drug release profiles in various regions of the rabbit's eye after injecting Bevacizumab.

FIG. 18: Drug release profiles of Bevacizumab in different regions of the eye after injection of Bevacizumab encapsulated liposomes.

FIG. 19: Drug release profiles of Bevacizumab in different regions of the eye after injection of liposome entrapped hydrogel.

FIG. 20: Drug release profiles of Bevacizumab in different regions of the eye after inserting an implant with liposome entrapped hydrogel.

FIG. 21: Implant with hydrogel in the rabbit's eye.

FIG. 22: Drug levels in anterior chamber in case of direct injection of avastin, avastin. liposomes, avastin liposomes in hydrogel and avastin liposomes in hydrogel filled in an implant.

FIG. 23: Drug levels in vitreous humor in case of direct injection of avastin, avastin liposomes, avastin liposomes in hydrogel and avastin liposomes in hydrogel filled in an implant.

FIG. 24: Drug levels retinal space in case of direct injection of avastin, avastin liposomes, avastin liposomes in hydrogel and avastin liposomes in hydrogel filled in an implant.

FIG. 25: Drug levels in anterior chamber when avastin liposomes in hydrogel filled in an implant were used to administer high doses of 6 mg and 9 mg of avastin.

FIG. 26: Drug levels in vitreous when avastin liposomes in hydrogel filled in an implant were used to administer high doses of 6 mg and 9 mg of avastin.

FIG. 27: Drug levels in retina when avastin liposomes in hydrogel filled in an implant were used to administer high doses of 6 mg and 9 mg of avastin.

FIG. 28: Oxidation pathway of L-DOPA.

FIG. 29(A)-29(C): UV-Vis spectroscopy of L-DOPA and oxidized products at pH7.4, 29(A) after 3 days; 29(B) after 35 days; 29(C) LC/MS base peak chromatograms of L-DOPA oxidation at pH 10 over a 4.5-hour time period.

FIG. 30: the base-peak chromatogram for the separation of L-DOPA oxidation products following this time period.

FIG. 31(A)-31(C): SAXS to determine the location of L-DOPA in the liposomes.

FIG. 32(A)-32(B): compares single intravitreal injection of Avastin v. liposomal Avastin in rabbit's eye.

FIG. 33: A drug delivery system (DDS) includes a bioabsorbable drug eluting implant inserted through a small incision, that is inflated after insertion with a drug-loaded hydrogel. Release of drugs over a period of 1 year were demonstrated.

### DETAILED DESCRIPTION

The following numbered embodiments are contemplated and are non-limiting:
1. An injectable formulation comprising:
   a hydrogel comprising one or more liposome compositions, and
   a therapeutic agent,
   wherein the therapeutic agent is present within the liposome composition, and wherein the therapeutic agent is additionally present within the hydrogel but outside of the liposome composition.
2. The injectable formulation of clause 1, wherein the liposome composition comprises a combination of DMPC, DOPE, DSPG, and cholesterol.
3. The injectable formulation of clause 2, wherein the liposome composition comprises a combination of DMPC, DOPE, DSPG, and cholesterol at a ratio selected from the group consisting of 70:0:0:30, 60:10:0:30, 60:0:10:30, 65:5:5:25, 60:5:5:30, and 65:0:10:25.
4. The injectable formulation of any one of clauses 1 to 3, wherein the liposome composition comprises a lipid bilayer, and wherein the lipid bilayer comprises a hydrophobic anti-oxidant.
5. The injectable formulation of any one of clauses 1 to 4, wherein the hydrogel is a biodegradable hydrogel.
6. The injectable formulation of any one of clauses 1 to 5, wherein the hydrogel entraps the one or more liposome compositions.
7. The injectable formulation of any one of clauses 1 to 6, wherein the hydrogel comprises a first liposome composition, a second liposome composition, and a third liposome composition.
8. The injectable formulation of clause 7, wherein the first liposome composition, the second liposome composition, and the third liposome composition are different.
9. The injectable formulation of clause 8, wherein the first liposome composition, the second liposome composition, and the third liposome composition have different release profiles for release of the therapeutic agent.
10. The injectable formulation of any one of clauses 7 to 9, wherein the first liposome composition, the second liposome composition, and the third liposome composition comprise a combination of DMPC, DOPE, DSPG, and cholesterol.
11. The injectable formulation of clause 10, wherein the first liposome composition comprises a combination of DMPC, DOPE, DSPG, and cholesterol at a ratio selected from the group consisting of 70:0:0:30, 60:10:0:30, 60:0:10:30, 65:5:5:25, 60:5:5:30, and 65:0:10:25.
12. The injectable formulation of clause 10, wherein the first liposome composition comprises a combination of DMPC, DOPE, DSPG, and cholesterol at a ratio of 60:10:0:30.
13. The injectable formulation of clause 10, wherein the second liposome composition comprises a combination of DMPC, DOPE, DSPG, and cholesterol at a ratio selected from the group consisting of 70:0:0:30, 60:10:0:30, 60:0:10:30, 65:5:5:25, 60:5:5:30, and 65:0:10:25.
14. The injectable formulation of clause 10, wherein the second liposome composition comprises a combination of DMPC, DOPE, DSPG, and cholesterol at a ratio of 65:5:5:25.
15. The injectable formulation of clause 10, wherein the third liposome composition comprises a combination of DMPC, DOPE, DSPG, and cholesterol at a ratio selected from the group consisting of 70:0:0:30, 60:10:0:30, 60:0:10:30, 65:5:5:25, 60:5:5:30, and 65:0:10:25.
16. The injectable formulation of clause 10, wherein the third liposome composition comprises a combination of DMPC, DOPE, DSPG, and cholesterol at a ratio of 60:5:5:30.
17. The injectable formulation of clause 10, wherein the first liposome composition comprises a combination of DMPC, DOPE, DSPG, and cholesterol at a ratio of 60:10:0:30, wherein the second liposome composition comprises a combination of DMPC, DOPE, DSPG, and cholesterol at a ratio of 65:5:5:25, and wherein the third liposome composition comprises a combination of DMPC, DOPE, DSPG, and cholesterol at a ratio of 60:5:5:30.
18. The injectable formulation of any one of clauses 7 to 17, wherein the hydrogel further comprises a fourth liposome composition, a fifth liposome composition, and a sixth liposome composition.
19. The injectable formulation of clause 18, wherein the fourth liposome composition, the fifth liposome composition, and the sixth liposome composition are different.
20. The injectable formulation of clause 19, wherein the fourth liposome composition, the fifth liposome composition, and the sixth liposome composition have different release profiles for release of the therapeutic agent.
21. The injectable formulation of any one of clauses 18 to 20, wherein the fourth liposome composition comprises a PEG moiety on its surface.
22. The injectable formulation of any one of clauses 18 to 20, wherein the fifth liposome composition comprises a PEG moiety on its surface.
23. The injectable formulation of any one of clauses 18 to 20, wherein the sixth liposome composition comprises a PEG moiety on its surface.
24. The injectable formulation of any one of clauses 18 to 20, wherein the fourth liposome composition, the fifth liposome composition, and the sixth liposome composition each comprise a PEG moiety on its surface.
25. The injectable formulation of any one of clauses 18 to 24, wherein the fourth liposome composition, the fifth liposome composition, and the sixth liposome composition comprise a combination of DMPC, DOPE, DSPG, and cholesterol.
26. The injectable formulation of clause 25, wherein the fourth liposome composition comprises a combination of DMPC, DOPE, DSPG, and cholesterol at a ratio selected from the group consisting of 70:0:0:30, 60:10:0:30, 60:0:10:30, 65:5:5:25, 60:5:5:30, and 65:0:10:25.
27. The injectable formulation of clause 25, wherein the fourth liposome composition comprises a combination of DMPC, DOPE, DSPG, and cholesterol at a ratio of 60:10:0:30.
28. The injectable formulation of clause 25, wherein the fifth liposome composition comprises a combination of DMPC, DOPE, DSPG, and cholesterol at a ratio selected from the group consisting of 70:0:0:30, 60:10:0:30, 60:0:10:30, 65:5:5:25, 60:5:5:30, and 65:0:10:25.
29. The injectable formulation of clause 25, wherein the fifth liposome composition comprises a combination of DMPC, DOPE, DSPG, and cholesterol at a ratio of 65:5:5:25.
30. The injectable formulation of clause 25, wherein the sixth liposome composition comprises a combination of DMPC, DOPE, DSPG, and cholesterol at a ratio selected from the group consisting of 70:0:0:30, 60:10:0:30, 60:0:10:30, 65:5:5:25, 60:5:5:30, and 65:0:10:25.
31. The injectable formulation of clause 25, wherein the sixth liposome composition comprises a combination of DMPC, DOPE, DSPG, and cholesterol at a ratio of 60:5:5:30.
32. The injectable formulation of clause 25, wherein the fourth liposome composition comprises a combination of DMPC, DOPE, DSPG, and cholesterol at a ratio of 60:10:0:30, wherein the fifth liposome composition comprises a combination of DMPC, DOPE, DSPG, and cholesterol at a ratio of 65:5:5:25, and wherein the sixth liposome composition comprises a combination of DMPC, DOPE, DSPG, and cholesterol at a ratio of 60:5:5:30.
33. The injectable formulation of any one of clauses 1 to 32, wherein the one or more liposome compositions have a size distribution between about 100 nm and about 300 nm.
34. The injectable formulation of any one of clauses 1 to 32, wherein the one or more liposome compositions have a size distribution between about 150 nm and about 300 nm.
35. The injectable formulation of any one of clauses 1 to 32, wherein the one or more liposome compositions have a size distribution between about 175 nm and about 300 nm.
36. The injectable formulation of any one of clauses 1 to 32, wherein the one or more liposome compositions have a size distribution between about 200 nm and about 300 nm.
37. The injectable formulation of any one of clauses 1 to 36, wherein the injectable formulation comprises a biodegradable polymer.
38. The injectable formulation of clause 36, wherein the biodegradable polymer is selected from the group consisting of polyvinylalcohol (PVA), segmented polyurethanes, polyureas, hyaluronic acid, methyl cellulose, HPMC, chitosan, PLGA, and collagen.
39. The injectable formulation of clause 36, wherein the biodegradable polymer is selected from the group consisting of polyacrylate, polyacrylamides, polyethylene oxide, and polyvinyl pyrrolidones (PVP).
40. The injectable formulation of clause 36, wherein the biodegradable polymer is a polyacrylate selected from the group consisting of poly(acrylic acid), poly(methacrylic acid), and poly(hydroxyethyl methacrylate).
41. The injectable formulation of any one of clauses 1 to 40, wherein the therapeutic agent is a retinal therapeutic agent.
42. The injectable formulation of any one of clauses 1 to 40, wherein the therapeutic agent is an anti-cancer therapeutic agent.
43. The injectable formulation of any one of clauses 1 to 40, wherein the therapeutic agent is an anti-Parkinson's disease therapeutic agent.
44. The injectable formulation of any one of clauses 1 to 40, wherein the therapeutic agent is a therapeutic agent for treating age related macular degeneration.
45. The injectable formulation of any one of clauses 1 to 40, wherein the therapeutic agent is an anti-VEGF antibody.
46. The injectable formulation of any one of clauses 1 to 40, wherein the therapeutic agent is bevacizumab.
47. The injectable formulation of any one of clauses 1 to 40, wherein the therapeutic agent is L-DOPA or an L-DOPA metabolite.
48. The injectable formulation of any one of clauses 1 to 40, wherein the therapeutic agent is selected from the group consisting of methadone, bupropion, buprenorphine, naloxone, naltrexone, or a combination thereof.
49. The injectable formulation of any one of clauses 1 to 40, wherein the therapeutic agent is naloxone.
50. The injectable formulation of any one of clauses 1 to 40, wherein the therapeutic agent is naltrexone.
51. The injectable formulation of any one of clauses 1 to 40, wherein the therapeutic agent is methadone.
52. The injectable formulation of any one of clauses 1 to 40, wherein the therapeutic agent is selected from the group consisting of antibodies, antibody fragments, anti-inflammatory drugs, siRNA, RNAi, miRNA, proteins, peptides, nucleic acids encoding polypeptides, immune checkpoint inhibitors, steroids, small molecules, receptor agonists, receptor antagonists, diagnostic agents, vitamins, anti-oxidants, gene editing complexes, chimeric, and fusion polypeptides.
53. The injectable formulation of any one of clauses 1 to 52, further comprising an excipient.
54. The injectable formulation of clause 53, wherein the excipient is selected from the group consisting of a hydrophobic anti-oxidant, a cryoprotectant, a drug stabilizer, a viscosity enhancer, and a mucoadhesive polymer.
55. The injectable formulation of clause 53, wherein the excipient is a formulation stabilizer.
56. A biodegradable implant comprising an expandable hollow matrix comprising pores and the injectable formulation of clause 1,
   wherein the expandable hollow matrix has a proximal end and a distal end,
   wherein the expandable hollow matrix has an interior defined by a wall of the expandable hollow matrix and extending between the proximal end to the distal end thereof, and
   wherein the expandable hollow implant has an unexpanded configuration and an expanded configuration.
57. The biodegradable implant of clause 56, wherein the biodegradable implant comprises a thermoplastic biodegradable polymer.
58. The biodegradable implant of clause 57, wherein the thermoplastic biodegradable polymer is selected from the group consisting of polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, polyamino acids, polymethyl vinyl ether, and copolymers, terpolymers, and any combination thereof selected from the group consisting of polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, polyamino acids, polymethyl vinyl ether, and copolymers, terpolymers, and any combination thereof.
59. The biodegradable implant of clause 56, wherein the injectable formulation comprises the injectable formulation of any one of clauses 1 to 55.
60. A method of administering the injectable formulation of clause 1 to a patient in need thereof, said method comprising the step of injecting a therapeutically effective amount of the injectable formulation to the patient.
61. The method of clause 60, wherein the injecting comprises a parenteral injection.
62. The method of clause 61, wherein the parenteral administration is an intravenous administration.
63. The method of clause 61, wherein the parenteral administration is an intramuscular administration.
64. The method of clause 61, wherein the parenteral administration is a subcutaneous administration.
65. The method of clause 61, wherein the parenteral administration is an intraocular administration.
66. The method of any one of clauses 60 to 65, wherein the injection is a single administration once per month.
67. The method of any one of clauses 60 to 65, wherein the injection is a single administration once per 6 months.
68. The method of any one of clauses 60 to 65, wherein the injection is a single administration once per 12 months.
69. The method of any one of clauses 60 to 65, wherein the injection is a single administration once per 18 months.
70. The method of clause 60, wherein the injectable formulation comprises the injectable formulation of any one of clauses 1 to 55.
71. A method of administering the biodegradable implant of clause 56 to a patient in need thereof, said method comprising the steps of inserting the biodegradable implant to the patient and injecting an injectable formulation into the biodegradable implant.
72. The method of clause 71, wherein the injecting comprises a parenteral injection.
73. The method of clause 72, wherein the parenteral administration is an intravenous administration.
74. The method of clause 72, wherein the parenteral administration is an intramuscular administration.
75. The method of clause 72, wherein the parenteral administration is a subcutaneous administration.
76. The method of clause 72, wherein the parenteral administration is an intraocular administration.
77. The method of any one of clauses 71 to 76, wherein the injection is a single administration once per month.
78. The method of any one of clauses 71 to 76, wherein the injection is a single administration once per 6 months.
79. The method of any one of clauses 71 to 76, wherein the injection is a single administration once per 12 months.
80. The method of any one of clauses 71 to 76, wherein the injection is a single administration once per 18 months.
81. The method of clause 71, wherein the biodegradable implant comprises the biodegradable implant of any one of clauses 56 to 59.

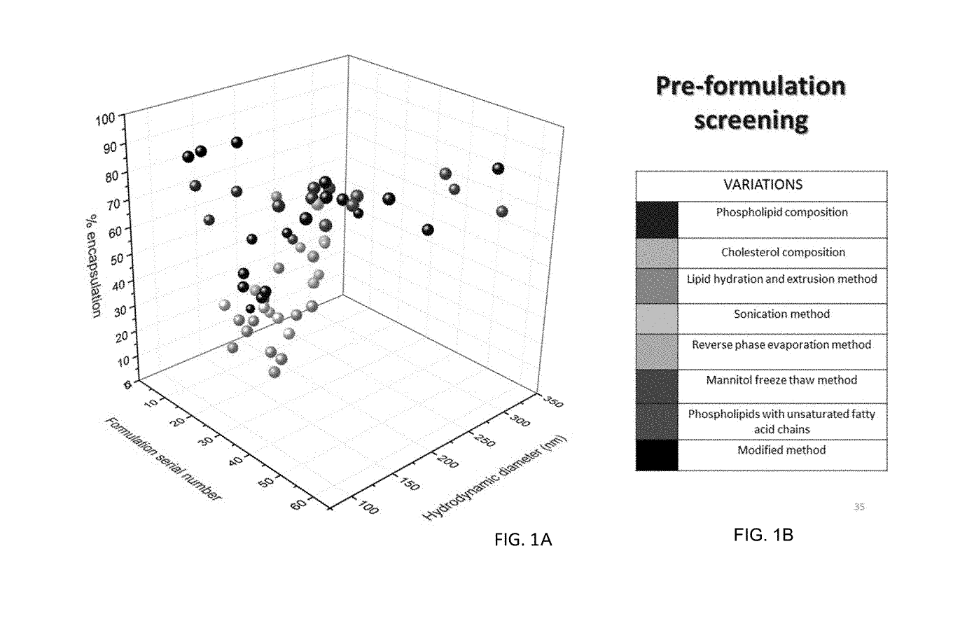

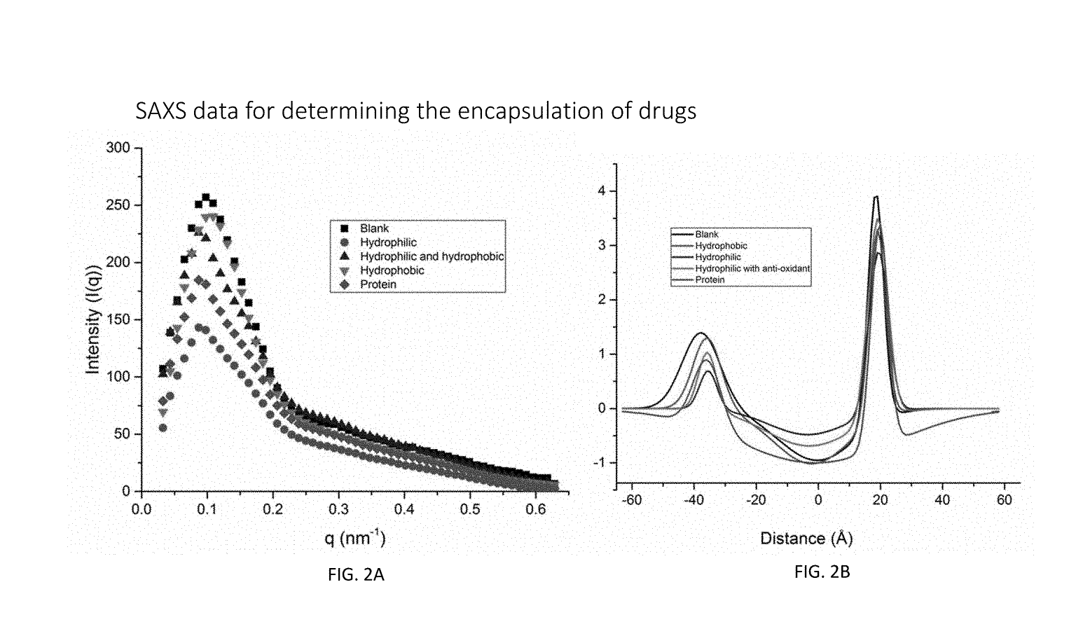

Preliminary formulation development was performed using fluorescent tagged IgG to determine various factors that affect the stability and drug release of the liposomes and the screening strategy can be seen in FIG. 1. Small Angle X-ray Scattering was performed to determine the exact location of the drug and encapsulation which can be seen in FIG. 2. From the particle size measurements, it was determined that liposomes with less concentration of cholesterol tend to be larger, on average, than liposomes with high cholesterol content. But, at the same time, higher concentration of cholesterol were observed to decrease the protein encapsulation efficiency of the liposomes. So, an optimum concentration of cholesterol is required to have stable bilayers as well as optimum release. Results shown in Tables 1-3, indicate that the use of 25-30% molar ratio of cholesterol with 70-75% molar ratio of total phospholipid content gives very stable formulations along with a sustained release. calorimetric studies support the data and have shown that excess unconjugated cholesterol above 30% in the composition appeared as a peak at around 157° C. indicative of crystalline cholesterol.

The effect of lipid composition on the size and stability of the liposomes have been examined by preparing liposomes with different molar ratios of phospholipids while maintaining the molar ratio of cholesterol constant. The liposomes with high concentrations of phosphatidylcholine gave an optimum size of 100-200 nm. On the other hand, the compositions with high concentration of phosphatidylglycerol (PG) were found to promote fusion and an overall increase in particle size. This can be attributed to the oxidation of unsaturated fatty acids on phosphatidylglycerol. From the data seen in Table 1, it may be inferred that lower the concentration of phosphatidylglycerol, lower the chance of oxidation of the liposomes, thereby, increasing the stability and shelf life. calorimetric studies revealed that the compositions with low or no phosphatidylglycerol content have more thermal stability as seen in Table 1. To overcome the disadvantage of oxidation and hydrolysis, hydrophobic anti-oxidants were incorporated within the formulations to be embedded in the lipid bilayers. A 3-fold difference in the particle size of liposomes was noted at different phospholipid and cholesterol concentrations in the presence of anti-oxidants. Lyophilization of liposomes promotes fusion and a concomitant increase in capture volume due to freeze fracture at extremely low temperatures. To avoid this problem, cryoprotectant sugars with non-eutectic behavior were used. Many groups have reported that even at high concentrations of the cryoprotectants, they could still observe about 8% of leakage per day. However, leakage was restricted by the disclosed material and methods to less than 0.5% by increasing the overall hydrophobicity of the liposome lipid bilayers by incorporating a hydrophobic anti-oxidant as seen in Table 1. A variety of sugars have been shown to act as protectants during dehydration/rehydration of liposomes. This protective ability can extend to prevent vesicle fusion and help in improved encapsulation of the marker within the liposomes. 175 mM of trehalose gives the best results in maintaining the stability during lyophilization. By adding the adjuvants and incorporating 8-10 freeze thaw cycles, the lipid hydration and extrusion method was modified to get stable liposomes with optimum particle size, higher encapsulation efficiency and slower release.

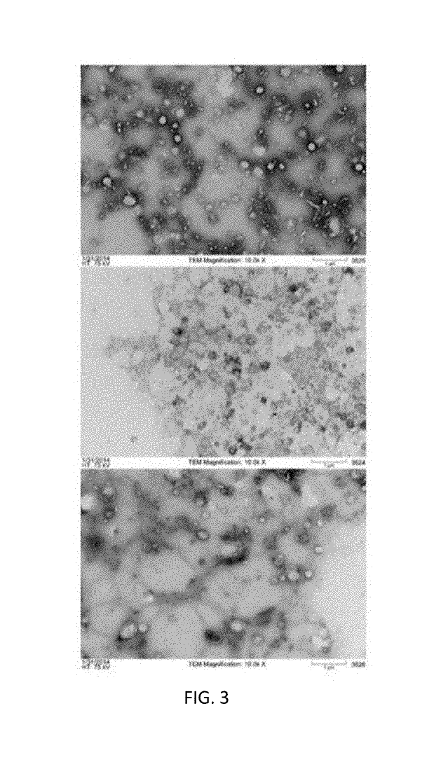

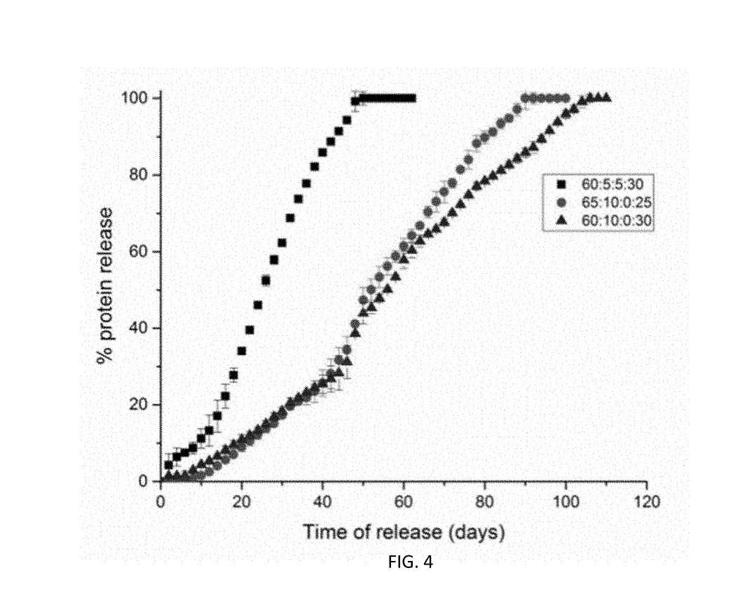

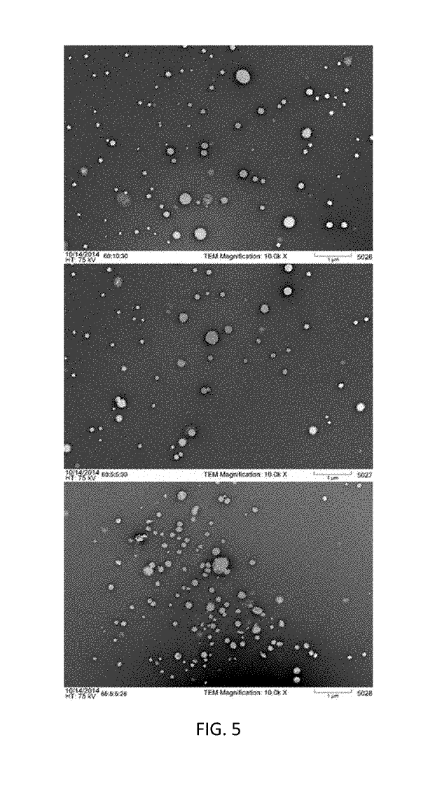

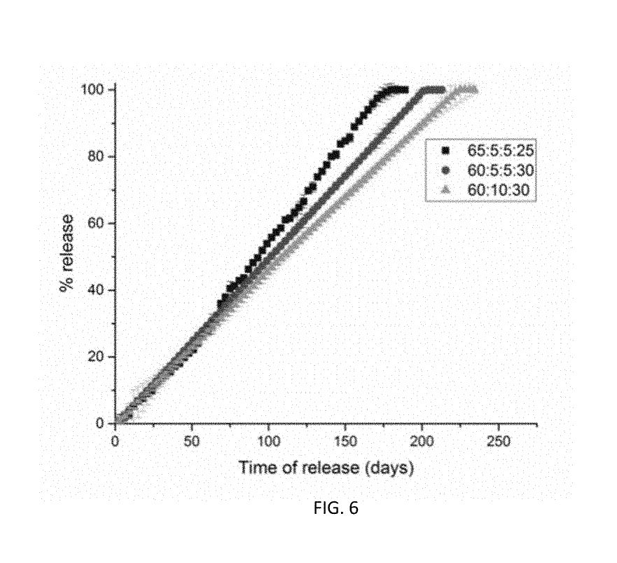

Using the information from all the preliminary studies based on particle size, encapsulation efficiency and time of release seen in FIGS. 3-6, formulations were downsized to three with the DMPC:DOPE:DSPG:cholesterol compositions of 60:10:0:30, 65:5:5:25 and 60:5:5:30 for encapsulating Bevacizumab. Using these compositions, two different kinds of liposomes were formulated—conventional and stealth liposomes. Stealth liposomes were prepared by surface modification of the liposomes using PEGylation method, where lipid bilayer of stealth liposomes is coated with long chains of polyethylene glycol, a hydrophilic polymer which forms a hydrated shell around the external surface of the lipid bilayer, thus creating a steric barrier preventing interactions with plasma proteins, opsonins, and cell surface receptors. Both conventional and stealth liposomes were found to be reasonably stable in terms of aggregation. To test the stability of the liposomes at various storage conditions, drug leakage studies were conducted as seen in Table 4. The drug leakage from conventional liposomes was 5-10 times faster than the stealth liposomes both at 4° C. and 37° C. in solution form. However, in both cases, reconstituted freeze-dried samples had much lesser leakage and hence is an ideal method of storage to increase the shelf life of the liposomal formulations.

Differential scanning calorimetry (DSC) was used to investigate the thermal stability as well as interaction between liposomes and entrapped protein. Thermal studies have shown that pure phospholipids, cholesterol and IgG gave sharp endotherms in a narrow temperature range and a combination of phospholipids as seen in the liposomes gave broader endotherms. The thermal behavior of the lipid bilayer phase transition was affected by the presence of adjuvants. This was clearly seen in both conventional and stealth liposomes as seen in Table 1. Another interesting feature is that the stealth liposomes produced endotherms approximately 5° C. higher than the conventional liposomes with same compositions. Others have postulated the increase might be due to lateral phase separation of PEG on the phospholipids caused by PEG chain entanglement and intra chain hydrogen bonds.

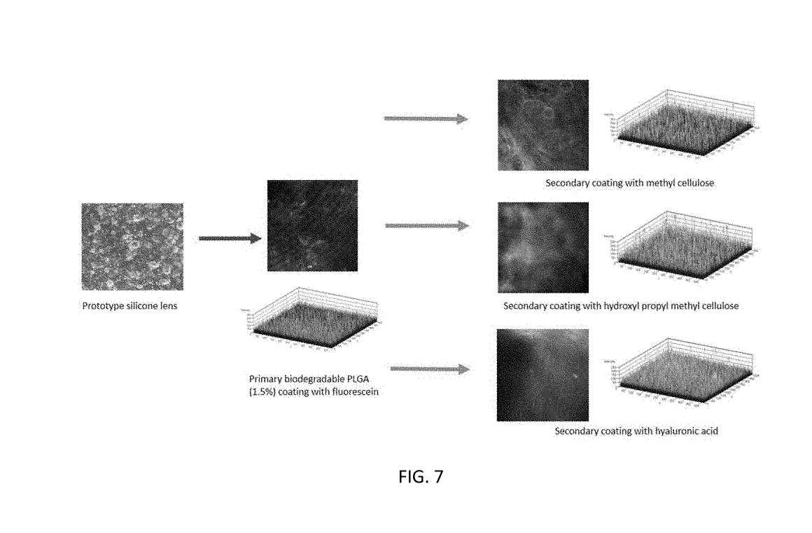

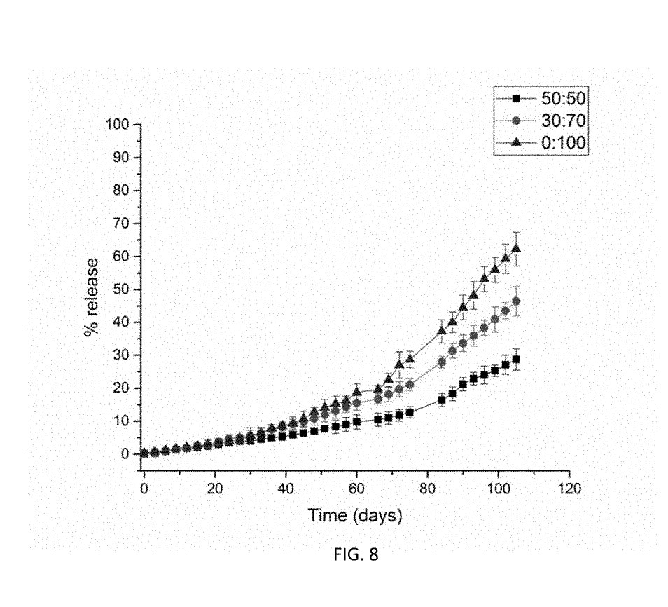

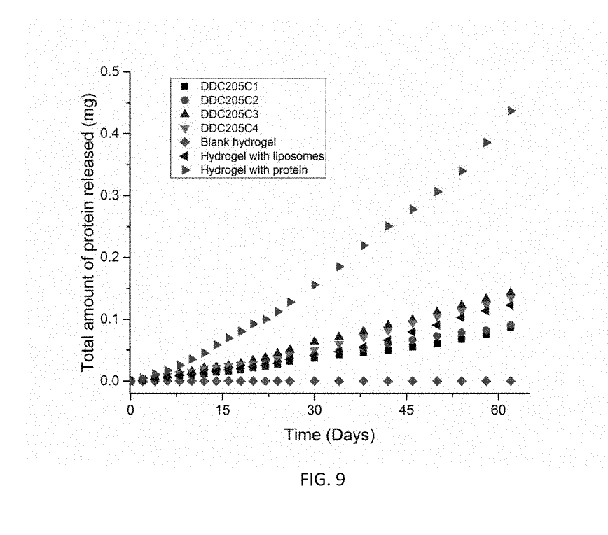

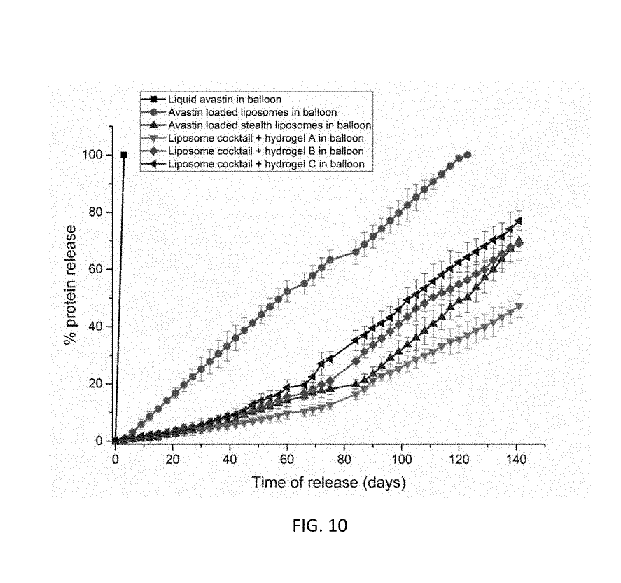

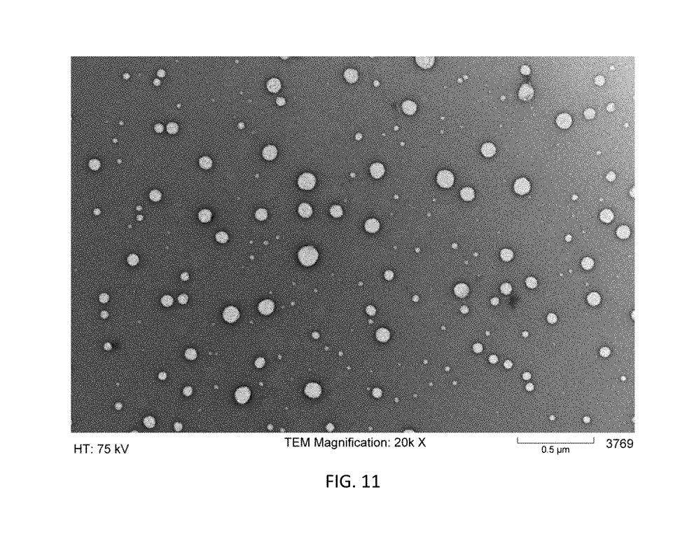

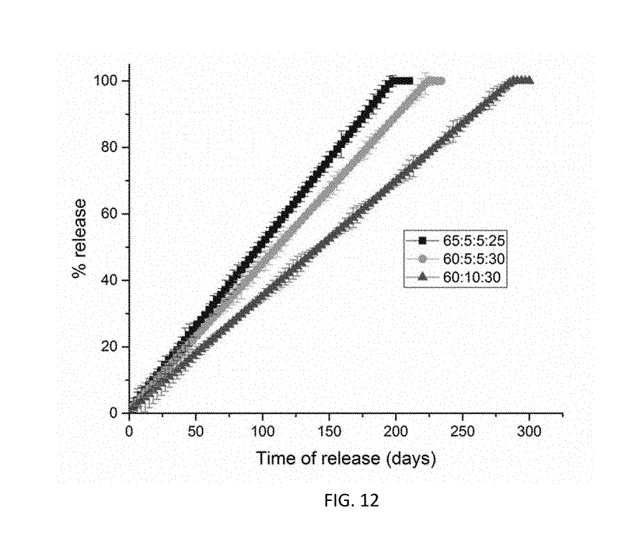

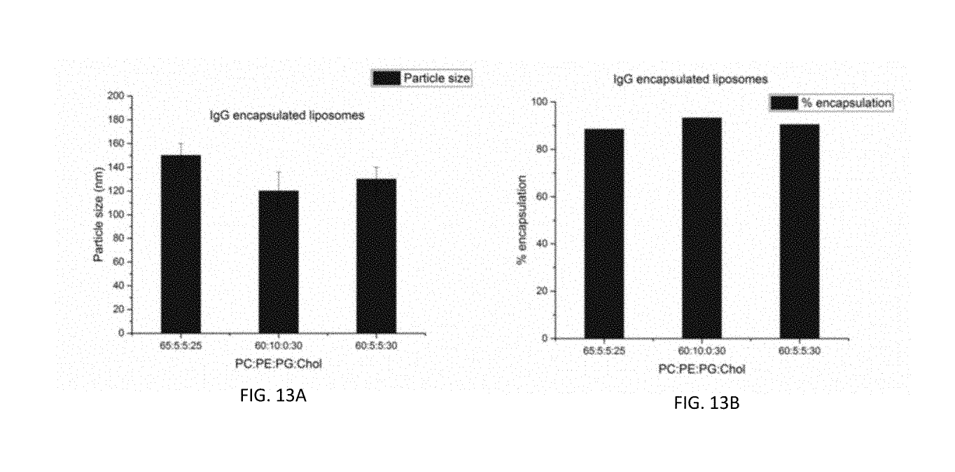

The drug release from the liposomes can be further controlled by incorporating the liposomes within a biodegradable hydrogel. The hydrogels can be coated as a thin layer on to an implant as shown in FIG. 7 or injected as a semisolid into the target site of administration. Different hydrogels were examined to determine the optimum release of Bevacizumab over a period of time as seen in FIGS. 8-10. Specifically FIG. 8 shows the drug release profiles when the hydrogels are made of varying concentrations of polyacrylic acid and hyaluronic acid. Various designs of hollow extendable implants made of biodegradable polymers were tested for the release of drug after injecting the liposome-hydrogel combination product. The results from using hollow extendable implants as reservoirs for the hydrogels has been shown in FIG. 9. A cocktail of liposomes of sizes varying from 100-300 nm at different phospholipid and cholesterol compositions were incorporated in to the hydrogel comprising hyaluronic acid and the drug release data is shown in FIG. 10. TEM images, drug release profiles, particle size and encapsulation efficiency of the liposome cocktail can be seen in FIGS. 10-13.

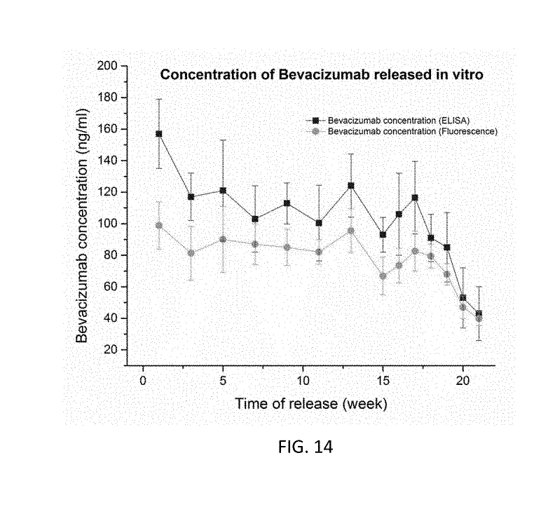

Increasing the stability of liposomes, while increasing the payload and preserving activity of encapsulated Bevacizumab in the process. Prototype formulations developed in vitro using IgG as a marker, were able to prolong the time of release while preventing protein degradation until release. Using the same molar compositions, Bevacizumab was formulated within the liposomes and in vitro drug release profiles and stability of the antibody were reported at regular intervals of time. Results from ELISA of the aliquots corroborate with the concentrations determined using UV-Vis spectrophotometry can be seen in FIG. 14. Based on the release profiles from different formulations, a cocktail of different compositions to provide an extended release drug delivery and to maintain the minimum effective concentration in the vitreous of the subject, is useful.

To determine the stability of the formulation, accelerated stability studies were performed at various storage conditions. Samples were aliquoted at different sampling points to ensure the thermal stability of the antibodies while in formulations. Potency of the antibodies was tested based on anti-VEGF activity of Bevacizumab in the aliquots using ELISA (data not shown). Presence of adjuvants like trehalose and beta carotene seem to maintain the stability of the antibody in liposomal solution. Trehalose is used in biologics as a cryoprotectant in the biopharmaceutical industry and hence, is a promising excipient even for liposomal drug delivery systems when administered in vivo.

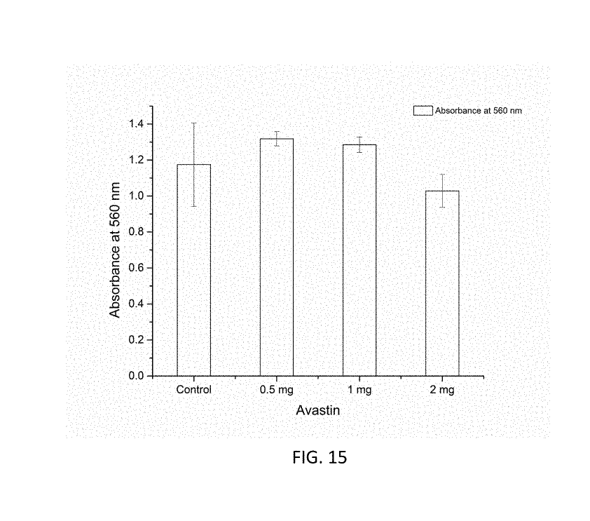

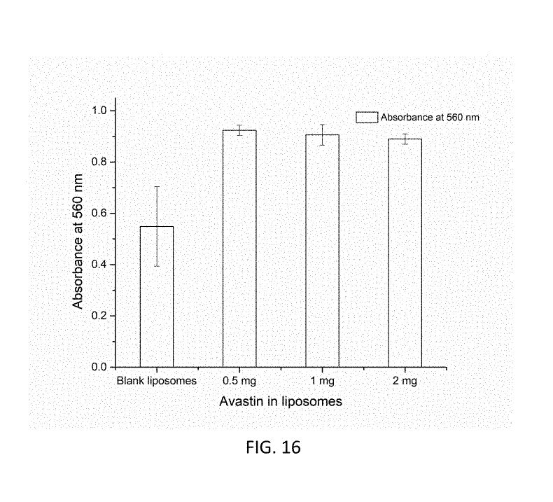

In some reports, 2.5 mg/ml of Bevacizumab exhibited cytotoxic effects on RPE cells. To assess effect of liposomes on the viability of both cell lines, MTT assay was performed. In case of cells treated with varying concentrations of Avastin, the cell viability decreased slightly when exposed to 2 mg of the antibody and the results were disclosed in FIG. 15. However, in case of cells treated with liposomes, the cell viability remained the same with liposomes encapsulating varying concentrations of Avastin as seen in FIG. 16. No cytotoxic effect of the vehicle (blank liposomes) could be observed. This observation is of importance considering that the volume of distribution in the eye for the antibody is very small compared to when administered systemically as a combination therapy for cancer, and hence, has to be regulated in terms of dose to be administered.

Overall, in vitro efficacy of the Bevacizumab loaded liposomes in terms of slow release was demonstrated. Anti-VEGF activity of the antibody was retained. Although administering high concentrations of the antibody can cause cytotoxic effects in vitro and in vivo, delivering the antibody through a vehicle controlling the slow release is a promising platform to reach the clinical setting.

### EXAMPLES

### Example 1: Rabbit Models

The efficacy of the liposomes and the combination product has been tested using rabbit models. The experimental design of this experiment involving Oryctolagus cuniculus (Dutch belted rabbits) was approved by the Northern Illinois University Institutional Animal Care and Use Committee (IACUC) and is as follows. Rabbits were divided into 4 groups and each rabbit was injected with the formulation in the right eye using left eye as control. Group 1 was injected with Bevacizumab and used as control. Group 2 was injected with Bevacizumab encapsulated liposomes. Group 3 was injected with Bevacizumab liposome entrapped hydrogel. Group 4 received an insert of the hollow extendable implant into which the Bevacizumab liposome entrapped hydrogel was injected.

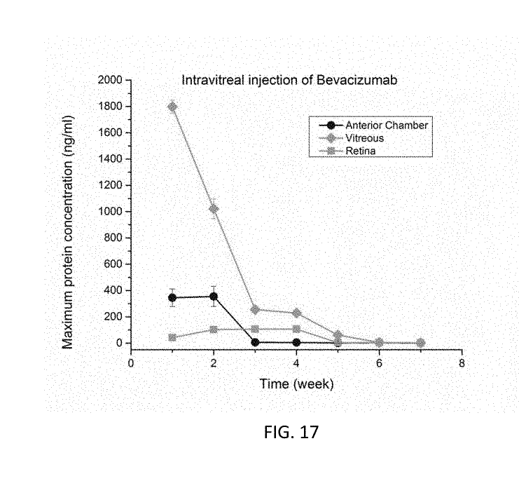

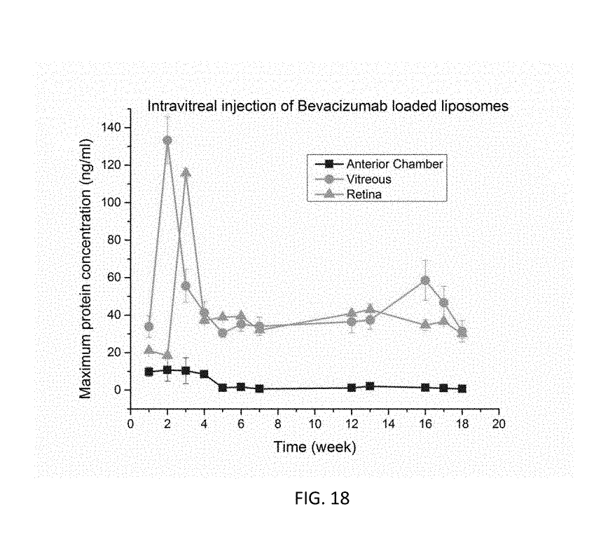

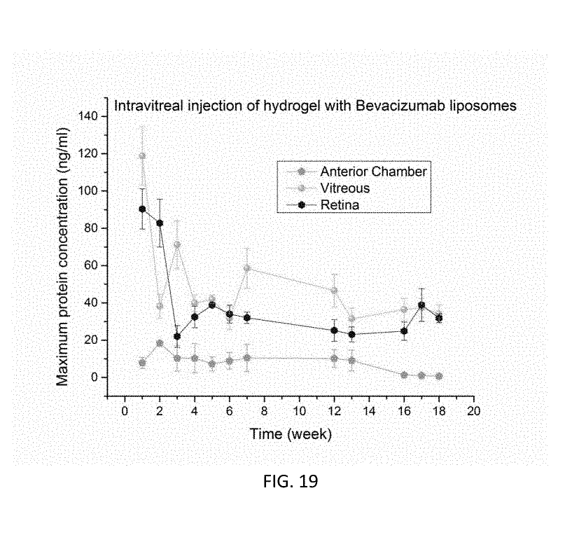

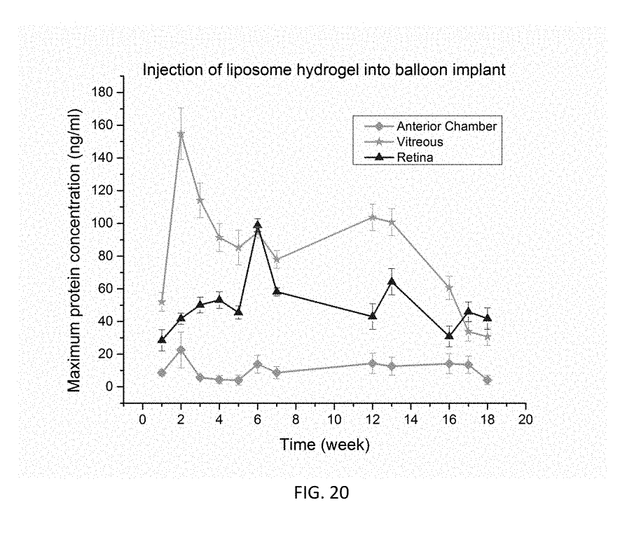

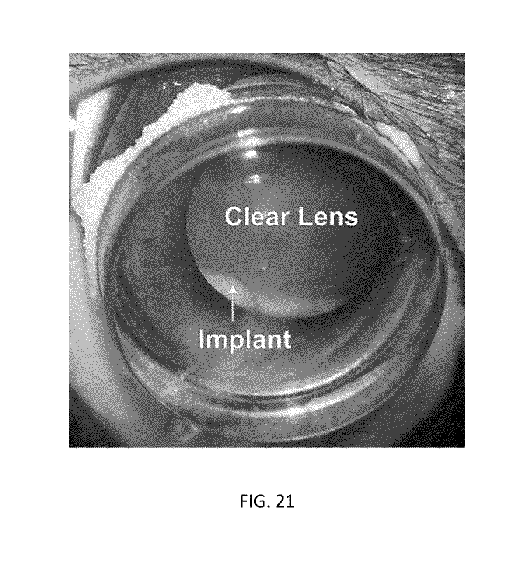

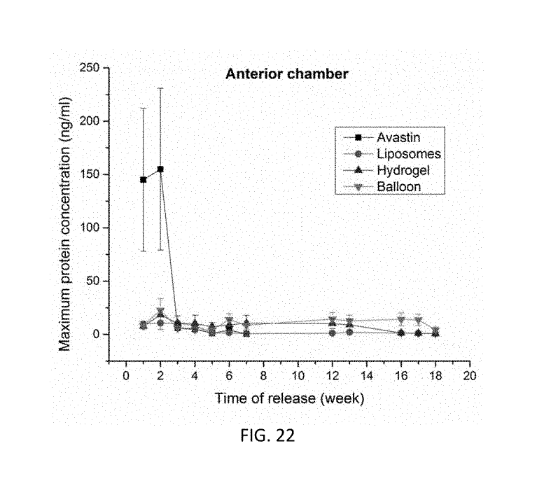

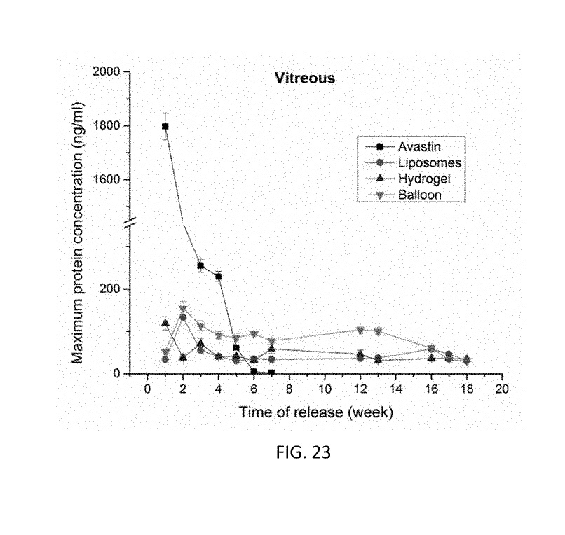

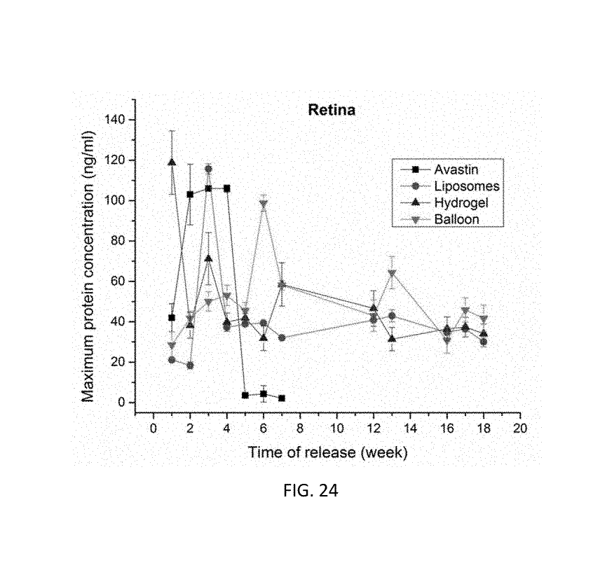

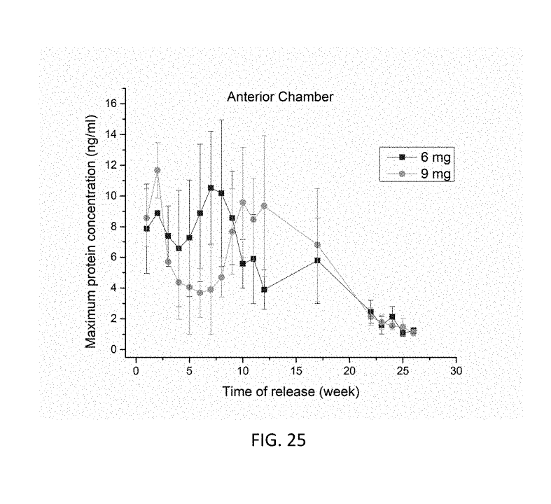

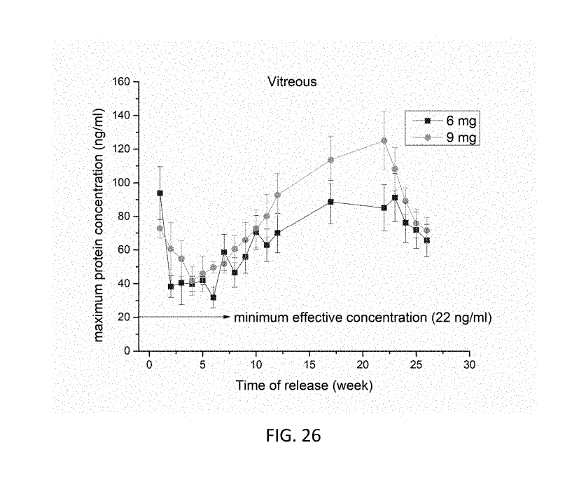

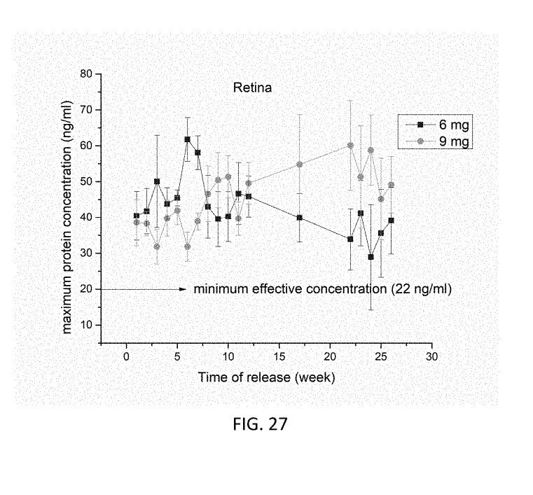

Drug release profiles in the rabbit's eye over a period of time was measured non-invasively using the Fluorotron. The excitation source irradiates, through a band-pass filter, an aperture Re which is imaged by the optical system on the retina as a 1.9×0.1 mm slit in the eye. Light re-emitted (reflection and fluorescence) by the fluorescent tag on the protein is sampled from the 1.9×0.1 mm slit, aligned to the excitation and defined by an aperture Rd (which is confocal to Re). Lens B is used to scan Rd and Re along the optical axis. The excitation and detection pupils are defined by the apertures Pc, located very close to lens C. These pupils are imaged anterior to the subject's cornea by the optics. The configuration of these pupils is in the plane of the subject's pupil minimizes contributions from the fluorescence outside of the measurement point by separating the excitation and detection paths. Another band-pass filter rejects reflected excitation light, and the fluorescence collected by aperture Rd is detected using a red-extended end-on photomultiplier tube selected for low (<100 count/sec.) dark noise. As the focus lens is driven forward the emission signal is continuously monitored from retina to cornea, giving an intensity output. Drug release profiles from Groups 1-4 can be seen in FIGS. 17-20 and the implant in the rabbit's eye can be seen in FIG. 21. Using the fluorotron, concentrations of the antibody in different regions of the rabbit's eye were measured. Groups 1-4 were compared in terms of drug concentrations at various intervals of time in the anterior chamber, vitreous humor and retinal space respectively in FIGS. 22-24. Dosage of drug administered into the eye were increased to 6 mg and 9 mg in the implant and the results can be seen in FIGS. 25-27.

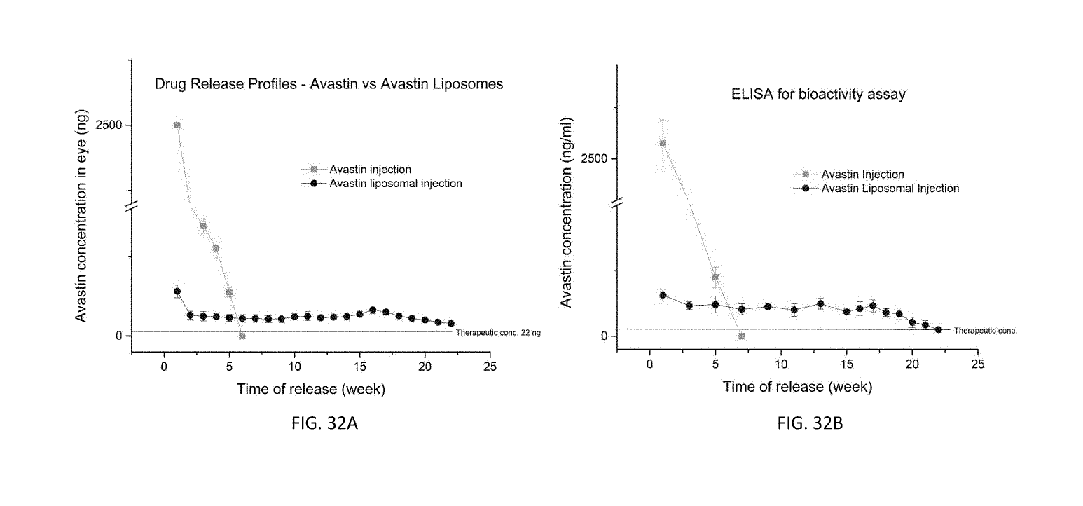

FIG. 32(A) and 32(B) shows drug release, profiles and ELISA bioactivity for Avastin compared to liposomal Avastin in rabbit's eye. Time of release of a therapeutic concentrate was 5 weeks compared to over 20 weeks. The biological activity of Avastin was confirmed. There was no toxicity as confirmed by cytotoxicity testis, histopathology; no protein aggregation.

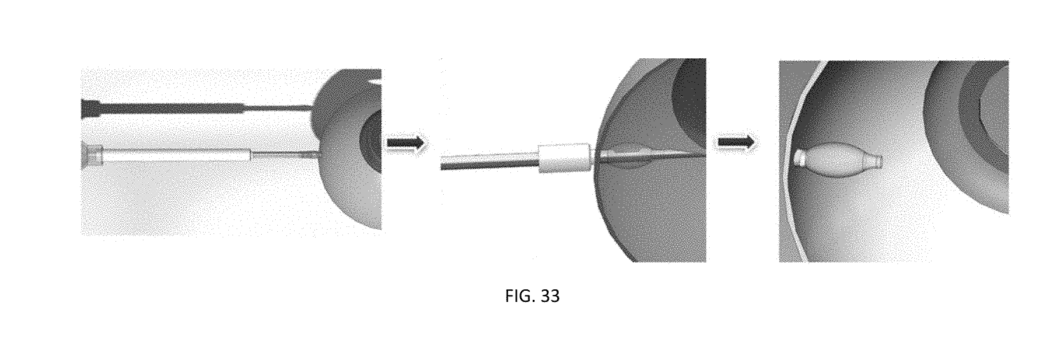

FIG. 33: Methods of delivery of drugs to the posterior of rabbit eyes through in vivo implantation in rabbits eyes demonstrated that after 5.5 months no toxicity was noted as result of the treatment. The lens and vitreous were clear, there was visible drug still in the implant, and no toxicity or inflammation was detected on histopathology.

### Example 2: Treatment of Parkinson's

A model drug delivery system for sustained release of Parkinson's drugs through subcutaneous injection is disclosed herein. L-DOPA oxidation products were characterized and the in vitro effects of these products in RPE cells were studied. Liposomal delivery systems for L-DOPA is contemplated in which the supplement was stabilized and the toxicity of oxidation products is reduced. Optimization of an efficient drug delivery system for L-DOPA to the target site is useful in the development of readily accessible long term treatment for Parkinson's disease with decreased side-effects and lower dose frequency.

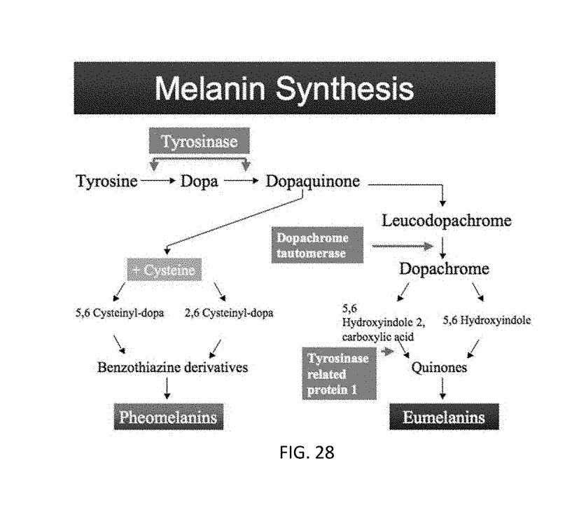

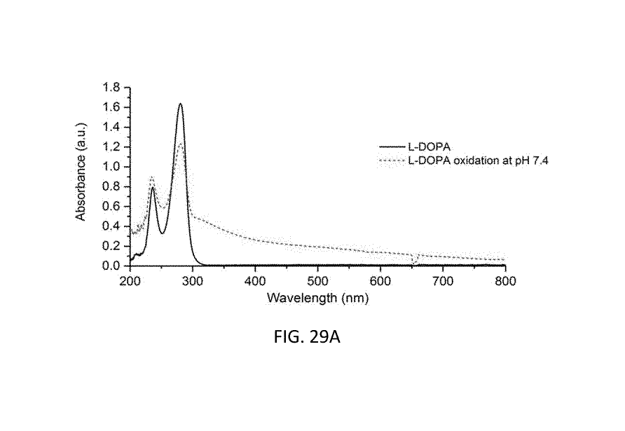

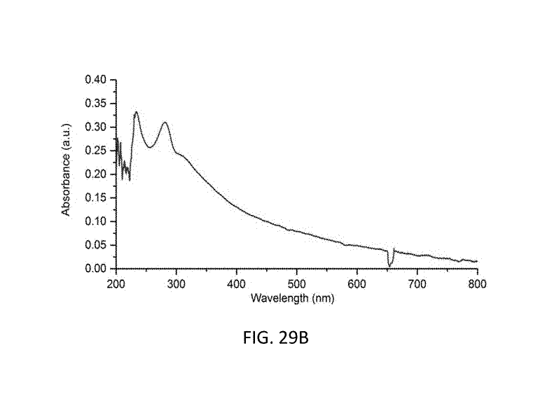

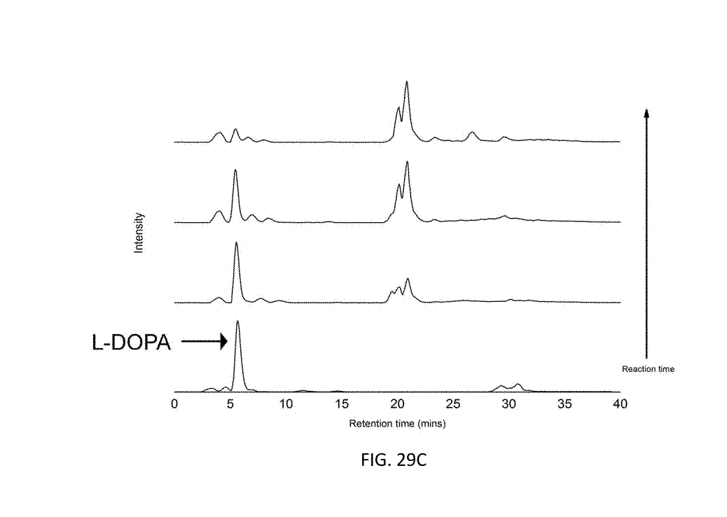

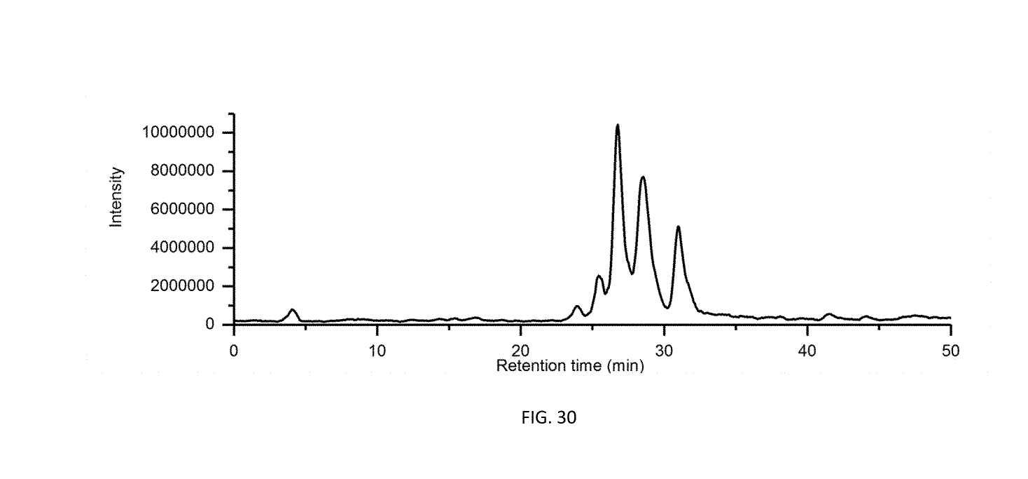

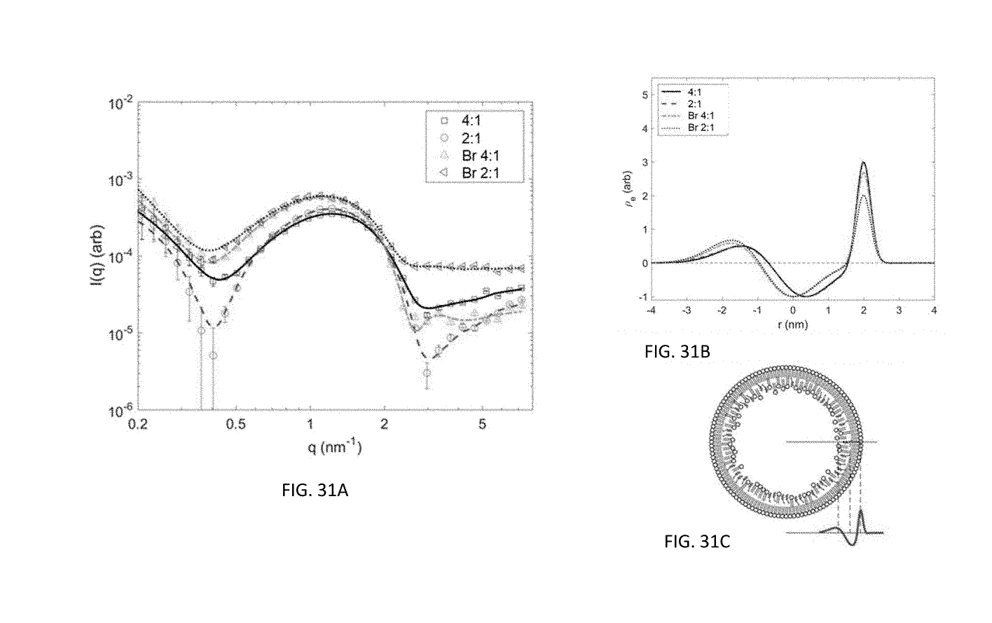

At pH 7.4, L-DOPA undergoes oxidation. FIG. 28 shows the oxidation pathway of L-DOPA. FIG. 29(A) shows the UV-Vis spectra of L-DOPA and oxidized L-DOPA after 3 days in PBS at pH 7.4. The solution was stored with minimal exposure to light. Under these conditions, L-DOPA does not undergo complete oxidation, as evidenced by the presence of characteristic absorbance maxima for the compound. FIG. 29(B) shows the absorbance of L-DOPA in pH 7.4 solution after 35 days. After this time period, there is substantial oxidation, however the starting material has not been fully oxidized. In order to accelerate the complete oxidation of L-DOPA starting material, the substance was reacted in at pH 10. FIG. 29(C) shows the LC/MS base peak chromatograms of L-DOPA oxidation at pH 10 over a 4.5-hour time period. After 24 hours, L-DOPA was completely consumed in the reaction and oxidation products were separated using LC/MS. FIG. 30 shows the base-peak chromatogram for the separation of L-DOPA oxidation products following this time period. Table 6 lists the retention time, m/z, and fragmentation data for L-DOPA oxidation products. These hydrophobic oxidation products were separated and collected for in vitro experiments with epithelialcells. FIG. 31(A)-31(C) is the SAXS data to show the localization of L-DOPA within the liposomes.

L-DOPA oxidation products may facilitate cell damage by inducing lysosome membrane permeability (LMP) and compromising lysosome function. It is known that lysosomal damage can initiate cell death pathways through several mechanisms such as release of cathepsins and caspase activation. Additionally, compromise to the lysosomal membrane can result in the leakage of proteases and undigested materials into the cytosolic fluid.

Exposure to L-DOPA oxidation products significantly increased the population of epithelial cells that exhibited LMP. These results were confirmed by testing lysosomal uptake and retention of Neutral Red dye in oxidation product fed cells. The hydrophobicity or possible detergent-like qualities of these oxidation products may explain increased membrane permeability in our in vitro system. The implication of these findings suggests toxicity of long-term L-DOPA oral supplements for treatment. The possibility of systemic oxidation of large doses of L-DOPA necessitates the need for controlled or sustained delivery and release of the drug to the target site for a more safe and effective outcome.

The effect of L-DOPA supplements on cells in vitro was probed. Stabilized delivery of L-DOPA to cells in vitro was effective using liposomes. Under oxidative stress conditions, liposomal delivery to cells stabilized L-DOPA supplements as evidenced by increased viability and active transport via lysosomes, versus L-DOPA added directly in media. Benefits of L-DOPA encapsulated in liposomes were sustained release drug delivery for Parkinson's disease. Furthermore, entrapping the liposomes within the hydrogel can be used as a strategy for controlled release.

### Example 3: Treatment of Substance Abuse

In another embodiment, the hydrogel was used for treating addiction. Naltrexone®, sold under the brand names ReVia® and Vivitrol® among others, is a medication primarily used to manage alcohol dependence and opioid dependence. In opioid dependence, it should not be started until people are detoxified. It is taken by mouth or by injection into a muscle. Effects begin within 30 minutes. A decreased desire for opioids however may take a few week. Naltrexone and its active metabolite 6β-naltrexol are competitive antagonists at the μ-opioid receptor (MOR), the κ-opioid receptor (KOR) to a lesser extent, and to a far lesser and possibly insignificant extent, at the δ-opioid receptor (DOR). The Ki affinity values of naltrexone at the MOR, KOR, and DOR have been reported as 0.0825 nM, 0.509 nM, and 8.02 nM, respectively, demonstrating a MOR/KOR binding ratio of 6.17 and a MOR/DOR binding ratio of 97.2. Naltrexone is metabolized in the liver mainly to 6β-naltrexol by the enzyme dihydrodiol dehydrogenase. Other metabolites include 2-hydroxy-3-methoxy-6β-naltrexol and 2-hydroxy-3-methoxy-naltrexone. These are then further metabolized by conjugation with glucuronide. The plasma half-life of naltrexone and its metabolite 6β-naltrexol are about 4 hours and 13 hours, respectively. As a result, a product for sustained release of naltrexone is desired to prevent addiction.

Naltrexone was encapsulated within the liposomes and the entrapped in a hydrogel. Dutch belted rabbits were used as animal models. For subcutaneous (SC) balloon implantation, the rabbits were administered with a general anesthetic. After the animal was under anesthesia, the surgical site was prepared as follows. 1 or 2% Lidocaine with Epinephrine was injected into the SC space to prevent pain. The skin was prepped with Betadine at the surgical site located superiorly within the animal's shoulder—lateral to the spine. Group 1 rabbits received 1 SC hollow extendable implant above the right shoulder, while Group 2 received a hydrogel with entrapped Naltrexone liposomesto evaluate dose-dependent pharmacokinetics. Next, a 2.4 mm incision was created with a special blade. A cannula was introduced into the SC space with a Trocar. A special hydrodissector was pushed through the cannula into the SC space. It was used to dissect a SC pocket by injecting saline under pressure. The excess fluid was aspirated out of the pocket. A pre-loaded implant was passed into the SC and inflated with the hydrogel with naltrexone-loaded liposome hydrogel. The positioning struts of the implant were trimmed flush with balloon and gently pushed under the skin. The skin incision was closed with a single 4-0 vicryl suture. Antibiotic ointment was applied to the incision for 1 week. The suture resorbs in about 2-3 weeks. Blood draws were performed at the prescribed intervals by the Animal Health Care Technician. The serum was centrifuged, frozen and stored for drug assay.

Naltrexone and its metabolite beta-naltrexol were monitored using HPLC. Drugs were separated on a C-18 column using 11.5% (vol/vol) acetonitrile and 0.4% (vol/vol) N,N,N,N-tetramethylethylenediamine within 20 min LC-MS/MS was performed using naltrexone-d (3) and 6beta-naltrexol-d (4) as internal standards. After protein precipitation, the chromatographic separation was performed on a C(18) column by applying a methanol gradient (5-100%, vol/vol) with 0.1% formic acid over 9.5 min. The HPLC/UV method was found to be linear for concentrations ranging from 2 to 100 ng/ml.

### Example 4: Aging Hydrogels

In another embodiment, the hydrogel technology is used for preparing an anti-aging hydrogel intended for topical use in a mammalian subject comprising a) 10 parts of 10 mg/ml crosslinked or non-crosslinked hyaluronic acid; b) 15 parts of 25 mg/ml hydrolyzed collagen; b) 75 parts of 65:10:25 molar ratio DMPC:DSPG:Cholesterol liposomes with entrapped retinol, vitamin C, palmitoyl oligopeptides, glutathione and beta carotene as an anti-oxidant system; c) anti-aging agents embedded in liposomes and hydrogel and c) other excipients to stabilize the formulation.

A 65:10:25 molar ratio DMPC:DSPG:Cholesterol was added in a sterile round bottom flask. To this, 10 ml of 2:1 ratio of methanol-chloroform mixture containing 1 μM beta carotene and 50 μM retinol was added to make a uniform organic phase. The solvent was removed by rotary evaporator to get a uniform film of lipid layer. This lipid layer was hydrated using the aqueous solution containing pre-determined amounts of Vitamin C, glutathione and palmitoyl oligopeptides. The liposome solution was allowed to hydrate overnight under dark and then, extruded 8-10 times to obtain a uniform particle size of around 100-150 nm. The final liposome solution was lyophilized under dark. The liposome powder is added to a mixture of hyaluronic acid-collagen hydrolysate gel to obtain a cream like consistency.

### Materials and Methods

1. Phospholipids

Phospholipids used for preparing liposomes like 1,2-dipalmitoyl-snglycero-3-phosphoethanolamine-N-[methoxy(polyethylene glycol)-2000](ammonium salt) (DPPE-PEG2000) and 1,2-dipalmitoyl-sn-glycero-3-phospho-(1′-rac-glycerol) (sodium salt) (DPPG) were purchased from Avanti Polar lipids, Inc. Milli-Q-water was produced via Millipore Milli-Q Plus Purepak 2 water purification system (EMD Millipore, Billerica, Mass.). Sodium phosphate, potassium phosphate, mannitol, trehalose, chloroform and high-performance liquid chromatography (HPLC) grade Methanol (MeOH) were purchased from Thermo Fisher Scientific (Pittsburgh, Pa., USA) Immunoglobulin G (IgG) from human serum, sodium chloride, cholesterol, beta-carotene, canthaxanthin, phosphatidylcholine and 5,6-carboxyfluorescein succinimidyl ester were from Sigma-Aldrich (St. Louis, Mo., USA).

2. Fluorescent Tagging of Protein

For easy detection of a model protein marker encapsulated within liposomes, proteins were labelled with fluorescein. IgG (a model protein for preliminary studies) and Bevacizumab (Avastin®) were tagged using NHS fluorescein (5,6-carboxyfluorescein succinimidyl ester). The protein (1 mg/ml) was dissolved in 20 mM sodium phosphate buffer with 0.15 M NaCl at pH 8.5. 4.7 μL of 15 mmol NHS-fluorescein solution in DMSO was added to the protein solution and incubated at room temperature for 2 hr. Unreacted NHS-fluorescein was removed by dialysis using 20 mM tris-glycine buffer at pH 8 for optimal results and accurate determination of the fluorophore-to-protein ratio. The absorbance of the labeled protein at 280 nm and 488 nm was measured to calculate the protein concentration and degree of labeling.

3. Preparation of Liposomes by a Modified Lipid Hydration and Extrusion Method

Different phospholipids and cholesterol were mixed at various molar ratios in a round bottom flask. To this, 10 ml of 2:1 ratio of methanol-chloroform mixture containing 1 μM anti-oxidant was added to make a uniform organic phase. The solvent was removed by rotary evaporator to get a uniform film of lipid layer. This lipid layer was hydrated using the protein solution in a pre-determined concentration of cryoprotectant sugar to get a final phospholipid concentration of 10 mg/ml. The dispersion was sonicated for 5 min in a bath sonicator. The liposome solution was extruded around 8-10 times to obtain a uniform particle size of 100-200 nm. The solution was then frozen at −70° C. for 30 min and then thawed to 40° C. for 20 min. The process was repeated at least 9-10 times and the final sample was lyophilized after removal of excess protein using gel permeation chromatography.

4. Particle Size Measurement

The particle size was measured by Dynamic light scattering (DLS) on a Brookhaven BI-200SM Research Goniometer and Laser Light Scattering System (5 mW He—Ne laser, λ=632 nm) using CONTIN software. To obtain the diffusion coefficient, the intensity correlation function must be analyzed. The hydrodynamic diameter and the particle size distribution were generated by the software. Accuracy of the data was determined based on polydispersity and baseline difference from the correlation curve. Particle size of the samples was measured at various stages and time intervals to determine the stability and shelf-life.

5. Encapsulation Efficiency

The change in fluorescence signal can be used to assess the membrane permeability. The extent of the leakage from an encapsulated liposome due to contact with a certain solute was determined from the relative fluorescence (% F) of the leaked protein and is calculated by equation:

$$Encapsulation efficiency = \frac{{F}_{T} - {F}_{0}}{{F}_{M} - {F}_{0}}$$

where, F_{T}—Fluorescence of liposomes after incubation with solute
F₀—Initial fluorescence due to dilution in an isomolar buffer
F_{M}—Maximal fluorescence after lysis by Triton X-100

6. Transmission Electron Microscopy for Imaging

Transmission electron microscopy was used for imaging the surface morphology of the liposomes. Briefly, a drop of a water-diluted suspension of the liposomes (about 0.05 mg/mL) was placed on a 200-mesh formvar copper grid (Electron Microscopy Sciences), allowed to adsorb and the surplus was removed by filter paper. A drop of 2% (w/v) aqueous solution of uranyl acetate was added and left in contact with the sample for 5 minutes. The surplus water was removed, and the sample was dried at room temperature before the vesicles were imaged with a TEM operating at an acceleration voltage of 200 KV.

7. Drug Release Studies

Different liposome compositions A1-A6 from Table 1 were prepared, lyophilized and stored separately until use. Three different compositions were used for preparing the extended release hydrogel. The liposome powder is added to a hydrogel matrix at 75 parts of liposomes to 25 parts of hydrogel to obtain a cream like consistency.

For fast release liposomes, phosphatidylethanolamine derivatives were substituted with phosphatidylserine.

In vitro drug release studies from antibody encapsulated liposomes in solution and entrapped in a hydrogel were performed using USP 4 dissolution apparatus (SOTAX Corporation). The flow rate was maintained at 1 ml/min. Aliquots were removed at regular intervals and the concentration of the drug released was screened using UV-Vis spectrophotometry. The concentration of protein in the solution was measured based on the absorbance from the fluorescent label using the equation:

$$Protein concentration ( M ) = \frac{{A}_{280} \times dilution factor}{{ɛ}_{protein}}$$

where, ε of IgG is 210,000 M⁻¹ cm⁻¹ and E of Bevacizumab is 1.7 cm ml/mg.

8. Accelerated Stability Studies

Accelerated stability testing of the formulations was performed by subjecting the samples to temperatures at 4° C. and 37° C. for a period of 30 days. During this period, aliquots collected on days 1, 14 and 30 were tested for protein released into the solution. % leakage was calculated based amount of protein in solution to total amount of protein encapsulated. The aliquots were then subjected to differential scanning calorimetry to determine thermal stability of the protein.

9. Differential Scanning Calorimetry for Thermal Stability Analysis

For thermal stability analysis of liposomes and encapsulated protein, two mg of standard lipids, protein and liposome samples were loaded in aluminum pans along with the standard reference aluminum in the differential scanning calorimeter (Shimadzu DSC 60). The thermal profiles were recorded between 10° C. and 180° C. at a scan rate of 15° C./min for three cycles.

10. ELISA for Determining Bevacizumab

Sandwich ELISA was employed for determining free Bevacizumab in the aliquots using Eagle Biosciences Bevacizumab ELISA Assay Kit. 100 μL of assay buffer was added into each of the wells. 50 μL of each 1:1000 diluted standard, and 1:1000 diluted aliquots were added into the respective wells of the microtiter plate and then covered with adhesive seal. The plates were then incubated for 60 min at room temperature. The incubation solution is aspirated, and the plate was washed 3 times with 300 μL of diluted wash buffer per well. After removing excess solution, 100 μL of Enzyme Conjugate (HRP-anti human IgG mAb) was pipetted into each well, covered and incubated for 30 min at room temperature. Washing the plate is repeated 3 times with 300 μL of diluted wash buffer per well. Finally, 100 μL of Ready-to-Use TMB Substrate Solution was added into each well and incubated for 15 min at room temperature. The substrate reaction is stopped by adding 100 μL of stop solution into each well. The color changes from blue to yellow and optical density (OD) is measured with a photometer at 450 nm within 15 min after pipetting the stop solution.

11. In Vitro Toxicity Studies

A. Bevacizumab

ARPE-19 cells purchased from American Type Culture Collection were used. Cells used for this study were from passages 10-35 and were maintained in Dulbecco's Modified Eagle Medium supplemented with 10% fetal bovine serum, 1.1% L-glutamine and 1.1% antibiotic/antimycotic. Cells were plated in 12-well plates with an initial seeding density of 5×10⁴ cells per ml in each plate. Cells were grown to confluency for 4 days prior to experimentation; 12-well plates were used for MTT assays. 1.5 ml of 25 mg/ml Avastin solution was sterile filtered to create a stock solution that was transferred to a sterile centrifuge tube. Each well of the 12 well plate had its media aspirated out and was then rinsed with buffer which was also aspirated out. Each well was then treated such that triplicates of 0.5 mg, 1 mg, 2 mg of avastin (bevacizumab) and liposomes with encapsulated avastin were being tested in the wells against controls. Enough media was added to each well to create a total volume of 1 ml in each well. The treated cells were incubated for 69 hours before the MTT assay. 1.3 ml of 10% MTT 5 mg/ml in PBS and 11.7 ml of 90% media (with phenol red) was used. MTT in PBS was added to the media in a centrifuge tube and then sterile filtered. The filtered mixture was then plated on the 12 well plate (1 ml per well). The plate was then incubated for 90 minutes. After incubation, the MTT media was aspirated. 1 ml of DMSO per well was added. The MTT assay was read at 560 nm.

B. L-DOPA

ARPE-19 cells purchased from American Type Culture Collection were used. Cells were maintained in Dulbecco's Modified Eagle Medium supplemented with 10% fetal bovine serum, 1.1% L-glutamine, and 1.1% antibiotic/antimycotic. Cells were plated on 24 well plates with an initial seeding density of 5×10⁴ cells/mL. Cells were grown to confluency for a period of 3-4 days prior to experimentation. Prior to irradiation, confluent cells were washed and layered with PBS. Cells were irradiated for 15 seconds with UV-C or subjected to dark treatment under the same conditions. Cells were positioned 5 inches under an 11 inch Ushio G8T5 germicidal UV-C bulb. PBS was gently aspirated and cells were supplemented with 10 μM L-DOPA or oxidation products from 10 μM L-DOPA starting material, in media. For liposome experiments, encapsulated L-DOPA concentration was matched to L-DOPA supplement given directly in cell media. Control cells were supplemented with vehicle or buffer encapsulated liposomes. Cells were incubated for either 1, 3, or 5 days with supplement post irradiation. MTT reagent was prepared at a concentration of 5 mg/mL in PBS and sterile filtered prior to use. After respective treatments, cells were incubated in 10% MTT in phenol red-free media solution for 90 minutes. MTT containing media was gently aspirated and formazan was dissolved in 500 μL of DMSO. Absorbance was measured at 570 nm using BioTek Synergy2 plate reader.

Cells were incubated with either 500 μM of L-DOPA or starting material concentration matched oxidation products for 24 hours. Media was aspirated and cells were washed twice with warm PBS. Cells were then incubated for 5 minutes with 0.5 μg/mL acridine orange in PBS or in PBS only. Cells were then washed 3 times with warm PBS and detached using TrypLE. Cells were harvested in PBS and washed by centrifugation. Cells were resuspended in PBS for flow cytometry analysis. Cells were sorted using a BD FACSCalibur flow cytometer. Intracellular fluorescence was measured using a 488-nm excitation laser and 530/30-nm emission bandpass filter or a 670 nm long pass emission filter. A total of 10,000 cells were counted per sample. Data was collected using CellQuest software and analyzed using FlowJo software.

12. LCMS Analysis

LC/MS analysis was performed using an LCQ Advantage Mass Spectrometer System with a Surveyor LC Pump and PDA Detector (Thermo Finnigan). Separations were performed on a 100 mm×3.0 mm Kinetex C18 column. Mobile phase was 0.1% formic acid in MeOH (B) balanced with water. Products were eluted using a gradient of 4-65% B (8-16 mins), 65-70% B (16-19 mins), 70-80% B (19-26 mins), and 80-95% B (26-50 mins) at a flow rate of 0.13 ml/min. Absorbance was monitored at 280 and 320 nm. For ESI-MS conditions, capillary temperature was 200° C. and spray voltage was set to 4.5 kV. For MS/MS and MS³ experiments collision energy was set at 28%. Full spectra were collected between m/z range of 200-1000.

13. Liposome Preparation

Liposomes were prepared using ethanol injection for encapsulating 100 μM L-DOPA, 100 μM fluorescein, or buffer as control. A 4:1 ratio of phosphatidylcholine to cholesterol was dissolved 1 mL of ethanol. 0.5 mL of this mixture was injected into 10 mL of solution at 4.29 mL/hour, with constant stirring. The final lipid concentration was 2.0 mg/mL. Argon gas was then bubbled through the mixture for 20 minutes, removing excess ethanol. Liposomes were then extruded 10 times using 0.2 and 0.1 μm polycarbonate filters, with nitrogen pressure maintained between 200-300 psi. Excess unencapsulated compounds in solution were removed from the solution by dialysis. Final liposome solutions were stored at 4° C. until use.

14. Encapsulation Efficiency

L-DOPA encapsulated liposomes were lysed with 1% of a 0.1% Triton-X solution. The mixture was vortexed for 30 seconds and allowed to rest for 5 minutes. Dynamic light scattering was performed to insure vesicles were no longer intact. UV-Vis measurements were taken of the lysed liposome solution and compared to the absorbance of a concentration matched L-DOPA solution. Percent encapsulation efficiency was determined by comparing the absorbance at λₘₐₓ. The average encapsulation efficiency of L-DOPA was 87±2%.

15. Hydrodynamic Diameter Measurement

Hydrodynamic diameter was measured by Dynamic light scattering (DLS) on a Brookhaven BI-200SM Research Goniometer and Laser Light Scattering System (5 mW He—Ne laser, λ=632 nm) using CONTIN software. The hydrodynamic diameter and the particle size distribution were generated by the software. Accuracy of the data was determined based on polydispersity and baseline difference from the correlation curve. Liposomes were 146±18 nm in diameter with polydispersity of 0.237±0.127 (mean±std dev, n=6).

16. Hydrogels

Hydrogel is further used for minimally-invasive diagnostic purposes. The hydrogel is employed for delivering diagnostic molecules like peptides, peptide hormones or probes for molecular imaging and disease diagnosis to a subject, wherein the hydrogel includes entrapped liposomes which encapsulate the diagnostic or imaging molecules; and hydrogel is injected by a suitable route of administration like subcutaneous, parenteral or intravitreal; and further screening the distribution of these diagnostic molecules assists in diagnosing a disease. A system is disclosed where the diagnostic molecules are either fluorescent labeled, radiolabeled, infrared labeled, magnetic labeled or and the fluorescence distribution in a specific combination is measured using a probe. For example, a preliminary study was performed using fluorescent tagged antibodies encapsulated within liposome-hydrogel complex and the release, localization as well as ocular bio-distribution was measured based on fluorescence measurement using a fluorotron as exemplified in FIGS. 17-20 and 22-27. Similar strategy can be developed for other diagnostic molecules based on the spectroscopic properties of the diagnostic molecules or by using detectable labels.

A biodegradable polymer is employed for preparing the hydrogels. Examples of hydrogel include polyvinylalcohol (PVA), segmented polyurethanes and suitable polyureas. Suitable hydrogels are non-biodegradable and porous. Preferably, the hydrogel has open-cell pores, allowing for ingrowth of the surrounding biological tissue. Other useful polymers include polyacrylate such as poly(acrylic acid), poly(methacrylic acid) and poly(hydroxyethyl methacrylate), polyacrylamides, polyethylene oxide and polyvinyl pyrrolidones (PVP). A variety of bioabsorbable/biodegradable polymers can be used to make the pores in the hydrogel matrix. Examples of biodegradable polymers include degradable polyesters such as polylactic acid, polyglycolic acid or their copolymers, eg. 50:50 PLA:PGA, the degradation profiles of which are well characterized.

Examples of other suitable biocompatible, bioabsorbable polymers include polymers selected from the group consisting of aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, biomolecules (i.e., biopolymers such as collagen, elastin, hyaluronic acid, soluble celluloses, bioabsorbable starches, etc.) and blends thereof. Aliphatic polyesters include, homopolymers and copolymers of lactide (which includes lactic acid, D-,L- and meso lactide), glycolide (including glycolic acid), ε-caprolactone, p-dioxanone (1,4-dioxan-2-one), trimethylene carbonate (1,3-dioxan-2-one), alkyl derivatives of trimethylene carbonate, 5-valerolactone, β-butyrolactone, γ-butyrolactone, ε-decalactone, hydroxybutyrate, hydroxyvalerate, 1,4-dioxepan-2-one (including its dimer 1,5,8,12-tetraoxacyclotetradecane-7,14-dione), 1,5-dioxepan-2-one, 6,6-dimethyl-1,4-dioxan-2-one 2,5-diketomorpholine, pivalolactone, α,α-diethylpropiolactone, ethylene carbonate, ethylene oxalate, 3-methyl-1,4-dioxane-2,5-dione, 3,3-diethyl-1,4-dioxan-2,5-dione, 6,8-dioxabicycloctane-7-one and polymer blends thereof.

Other exemplary bioabsorbable, biocompatible elastomers include elastomeric copolymers of ε-caprolactone and glycolide (including polyglycolic acid) with a mole ratio of ε-caprolactone to glycolide of from about 35 to about 65 to a mole ratio of about 65 to about 35, more preferably from a mole ratio of about 45 to about 55 to a mole ratio of about 35 to about 65; elastomeric copolymers of ε-caprolactone and lactide (including L-lactide, D-lactide, blends thereof, and lactic acid polymers and copolymers) where the mole ratio of ε-caprolactone to lactide is from about 35 to about 65 to a mole ratio of about 65 to about 35 and more preferably from a mole ratio of about 45 to about 55 to a mole ratio of about 30 to about 70 or from a mole ratio of about 95 to about 5 to a mole ratio of about 85 to about 15; elastomeric copolymers of p-dioxanone (1,4-dioxan-2-one) and lactide (including L-lactide, D-lactide, blends thereof, and lactic acid polymers and copolymers) where the mole ratio of p-dioxanone to lactide is from about 40 to about 60 to a mole ratio of about 60 to about 40; elastomeric copolymers of ε-caprolactone and p-dioxanone where the mole ratio of ε-caprolactone to p-dioxanone is from about 30 to about 70 to a mole ratio of about 70 to about 30; elastomeric copolymers of p-dioxanone and trimethylene carbonate where the mole ratio of p-dioxanone to trimethylene carbonate is from about 30 to about 70 to a mole ratio of about 70 to about 30; elastomeric copolymers of trimethylene carbonate and glycolide (including polyglycolic acid) where the mole ratio of trimethylene carbonate to glycolide is from about 30 to about 70 to a mole ratio of about 70 to about 30; elastomeric copolymers of trimethylene carbonate and lactide (including L-lactide, D-lactide, blends thereof, and lactic acid polymers and copolymers) where the mole ratio of trimethylene carbonate to lactide is from about 30 to about 70 to a mole ratio of about 70 to about 30, and blends thereof.

17. Embodiment of Drugs Suitable for Use

The drug can be a mixture of [at least three or more of antibodies, antibody fragments, anti-inflammatory drugs, siRNA, RNAi, miRNA, proteins, peptides, nucleic acids, immune checkpoint inhibitors, steroids, small molecules, receptor agonists, receptor antagonists, diagnostic agents, fusion polypeptides; wherein the nanoparticles can be solid lipid nanoparticles, liposomes, mucoadhesive nanoparticles, polymer nanoparticles, dendrimers, cyclodextrins, niosomes or a combination.] The nanoparticles further include a cocktail of different compositions to obtain varying drug release profiles based on surface modifications. [The nanoparticles are in the size range of 100-150 nm, having an encapsulation efficiency of at least 85-92%, have high load capacity, capable of encapsulating hydrophilic, amphiphilic and hydrophobic drugs. The excipients include hydrophobic anti-oxidants, cryoprotectants, small molecules that stabilize the drugs, viscosity enhancers and mucoadhesive polymers.]

18. A Method of Implanting a Biodegradable Implant into a Patient's Body

The method includes (a) establishing an expandable/extendable hollow implant with pores ranging in nanometer to micrometer size formed from a slow biodegrading composition with a degradation time of 6-12 months and having a proximal end and a distal end, the expandable hollow implant having an interior defined by a wall of the expandable hollow implant and extending between the proximal end to the distal end thereof, wherein the hollow implant has an unexpanded configuration and an expanded configuration; (b) inserting the expandable hollow implant in the unexpanded configuration subcutaneously into the patient's body to a predetermined depth; (c) subsequent to insertion of the expandable hollow implant to a desired location in the patient's body, inflating the expandable hollow implant with a liposome hydrogel complex transported from the central section chamber formed in the central section of a tubularly shaped injection member to the interior of the expandable hollow implant through the at least one injection opening formed through the wall of the central section of the tubularly shaped injection member; (d) subsequent to inflating the expandable hollow implant with the hydrogel, withdrawing the tubularly shaped injection member from the interior of the expanded hollow implant and the patient's body; (f) securing the expanded hollow implant within the patient's body; and (g) wherein the volume of the implant in expanded state accommodates 0.1 ml to 2 ml based on the site and route of administration.

Also disclosed is the thermoplastic biodegradable polymer of the implant which may be polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, polyamino acids, polymethyl vinyl ether, and copolymers, terpolymers, and any combination thereof.

For an implant, suitable thermoplastic polymers include polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, polyamino acids, polymethyl vinyl ether, and copolymers, terpolymers, and any combination thereof.

For an implant, suitable biologically-active agents include anti-inflammatory agents, ocular drugs, anti-neurodegenerative agents, antibacterial agents, antiparasitic agents, antifungal agents, antiviral agents, anti-neoplastic agents, analgesic agents, anaesthetics, vaccines, central nervous system agents, growth factors, hormones, antihistamines, osteoinductive agents, cardiovascular agents, anti-ulcer agents, bronchodilators, vasodilators, birth control agents and fertility enhancing agents.

The drug delivery system benefits patients who require long term therapy to treat neurodegenerative diseases or addiction. The drugs which are intended for sustained release over a period of time include Methadone (Dolophine, Methadose), bupropion, buprenorphine (Suboxone, Subutex, Probuphine, Sublocade), naloxone, naltrexone (Vivitrol), Cholinesterase inhibitors, Donepezil, Galantamine, Rivastigmine, Memantine, L-dopa, 1-dopa metabolites, dopamine, dopamine agonists and antagonists, serotonin receptor agonists and antagonists, DHICA, melanin, melanin precursors and melanin like compounds.

19. Drug Delivery

The drug delivery system is useful in various fields of medicine to tackle a plethora of problems, disorders and conditions.

The drug delivery system can release minimum effective drug concentrations over a period of 12-18 months.

Bevacizumab is currently approved for use in colorectal cancer. The disclosed methods and compositions can be used in other embodiments to deliver other cancer drugs.

The list of cancer drugs can be selected from the list consisting of and not restricted to anti-VEGF antibodies, Bevacizumab (Avastin), Ranibizumab (Lucentis), Pegaptanib (Macugen), Aflibercept (Eylea), monoclonal antibodies, polyclonal antibodies, fusion polypeptides, fusion antibodies, Atropine, Flurbiprofen, Physostigmine, Azopt, Erbitux, Gentamicin, Abemaciclib, Abiraterone Acetate, Abitrexate (Methotrexate), Abraxane (Paclitaxel Albumin-stabilized Nanoparticle Formulation), ABVD, ABVE, ABVE-PC, AC, Acalabrutinib, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin (Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avelumab, Axicabtagene Ciloleucel, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bexarotene, Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Calquence (Acalabrutinib), Campath (Alemtuzumab), Camptosar (Irinotecan Hydrochloride), Capecitabine, CAPDX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt, Dexamethasone, Dexrazoxane Hydrochloride, Dinutuximab, Docetaxel, Doxil, Doxorubicin Hydrochloride, DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Everolimus, Evista (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), Recombinant HPV Bivalent Vaccine, Recombinant HPV Nonavalent Vaccine, Recombinant HPV Quadrivalent Vaccine, Hycamtin (Topotecan Hydrochloride), Hydrea (Hydroxyurea), Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride, Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lutathera (Lutetium Lu 177-Dotatate), Lutetium (Lu 177-Dotatate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R—CHOP, R—CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thioguanine, Thiotepa, Tisagenlecleucel, Tolak (Fluorouracil—Topical), Topotecan Hydrochloride, Toremifene, Torisel (Temsirolimus), Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, Valrubicin, Valstar (Valrubicin), Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yescarta (Axicabtagene Ciloleucel), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), Zytiga (Abiraterone Acetate) or a combination of drugs.

TABLE 1
Liposome Composition

| S. No. | Composition | Molar ratios |
|---|---|---|
| A1 | DMPC:DSPG:DOPE:Chol | 65:5:5:25 |
| A2 | DMPC:DSPG:DOPE:Chol | 60:5:5:30 |
| A3 | DMPC:DSPG:DOPE:Chol | 60:0:10:30 |
| A4 | DMPC:DSPG:DPPE-PEG2000:Chol | 65:5:5:25 |
| A5 | DMPC:DSPG:DPPE-PEG2000:Chol | 60:5:5:30 |
| A6 | DMPC:DSPG:DPPE-PEG2000:Chol | 60:0:10:30 |

TABLE 2
DSC melting temperatures as a measure of thermal stability.

| Sample | Composition | Melting temperature, Tₘ | |
|---|---|---|---|
| Protein | IgG | ~59° | C. |
| | Bevacizumab | ~63.7° | C. |
| Lipids | DMPC | ~42° | C. |
| | DOPE | ~63° | C. |
| | DSPG | ~40° | C. |
| | Cholesterol | ~154° | C. |
| Conventional | 60:5:5:30 | ~87° | C. |
| liposomes | 65:5:5:25 | ~65° | C. |
| | 60:10:0:30 | ~98.8° | C. |
| Stealth | 60:5:5:30 | ~92° | C. |
| liposomes | 65:5:5:25 | ~70.4° | C. |
| | 60:10:0:30 | ~104.3° | C. |
| PC- cholesterol | 80:20 | ~73° | C. |
| liposomes | 70:30 | ~89° | C. |

| | | |
|---|---|---|
| with varying | 60:40 | ~87° C., ~157° C. |
| cholesterol | 50:50 | ~82° C., ~157° C. |
| content | 40:60 | ~76° C., ~157° C. |

TABLE 3
Particle size, encapsulation efficiency and time of protein
release from different formulations with various molar
concentrations of phospholipids and cholesterol.

| PC:PE:PG:Chol | Particle | Encapsulation | Time of |
|---|---|---|---|
| molar ratio | size (nm) | efficiency (%) | release (days) |
| 70:0:0:30 | 168 ± 32 | 39.2 ± 2.1 | 27 ± 2 |
| 60:10:0:30 | 231 ± 13 | 52.2 ± 5.6 | 37 ± 4 |
| 60:0:10:30 | 385 ± 23 | 43.5 ± 3.6 | 31 ± 6 |
| 60:5:5:30 | 278 ± 11 | 49.7 ± 2.1 | 35 ± 3 |
| 65:5:5:25 | 301 ± 19 | 34.8 ± 4.2 | 24 ± 6 |

TABLE 4
Particle size, encapsulation efficiency and time of protein release
from different formulations with various molar concentrations
of phospholipids and cholesterol after PEGylation and incorporating
beta-carotene and trehalose in the composition

| PC:PE-PEG2000:PG:Chol | Particle | Encapsulation | Time of |
|---|---|---|---|
| composition | size (nm) | efficiency (%) | release (days) |
| 70:0:0:30 | 120 ± 32 | 60.2 ± 2.1 | 57 ± 2 |
| 60:10:0:30 | 125 ± 21 | 90.1 ± 4.2 | 108 ± 4 |
| 60:0:10:30 | 175 ± 23 | 73.5 ± 3.6 | 71 ± 6 |
| 60:5:5:30 | 120 ± 11 | 93 ± 2.1 | 95 ± 3 |
| 65:5:5:25 | 152 ± 12 | 89 ± 3.2 | 84 ± 6 |

TABLE 5
Stability study based on percentage leakage of the protein at 4° C. and 37° C.

| | | % Drug leakage | | | % Drug leakage | | |
|---|---|---|---|---|---|---|---|
| | | at 4° C. | | | at 37° C. | | |
| Sample | PC:PE:PG:Chol | Day 1 | Day 14 | Day 30 | Day 1 | Day 14 | Day 30 |
| Conventional | 60:5:5:30 | 0.31 | 2.35 | 6.15 | 1.73 | 15.3 | 23.5 |
| liposomes | 65:5:5:25 | 0.35 | 2.67 | 6.5 | 2.19 | 17.1 | 25.2 |
| | 60:10:0:30 | 0.25 | 1.98 | 5.4 | 1.57 | 14.2 | 18.3 |
| Stealth | 60:5:5:30 | 0.04 | 0.52 | 1.23 | 0.28 | 2.84 | 5.3 |
| liposomes | 65:5:5:25 | 0.03 | 0.63 | 1.41 | 0.22 | 2.75 | 5.8 |
| | 60:10:0:30 | 0.05 | 0.48 | 1.18 | 0.26 | 2.41 | 5.1 |

TABLE 6
Retention time and MS, MS/MS, and MS³ ions for
separation of L-DOPA oxidation products.

| Retention time | m/z | MS/MS fragments | MS³ fragments |
|---|---|---|---|
| 23.9 minutes | 383 | 365 355 337 | 338 320 309 |
| | | 309 265 | 295 265 248 |
| | | | 219 202 |
| 25.4 minutes | 365 | 347 319 301 | 329 319 301 |
| 26.8 minutes | 462 | 444 418 400 | 400 372 329 |
| | | 372 | |
| 28.6 minutes | 490 | 472 446 402 | 460 428 400 |
| | | 384 | 384 366 356 |
| 30.9 minutes | 341 | 313 327 | — |

### PUBLICATIONS

These publications are incorporated by reference to the extent they relate materials and methods disclosed herein.
Bailey et al., “Synthesis of Polymerized Vesicles with Hydrolyzable Linkages”, Macromolecules, 25:3-11 (1992).
Klibanov et al., “Activity of Amphipathic Poly(ethylene glycol) 5000 to Prolong the Circulation Time of Liposomes Depends on the Liposome Size and Is Unfavorable for Imnunolipososome Binding to Target”, Biochimica et Biophysica Acta, 1062:142-148 (1991).
Lasic et al., “Sterically Stabilized Liposomes: a Hypothesis on the Molecular Origin of the Extended Circulation Times”, Biochimica et Biophysica Acta, 1070:187-192 (1991).
Torchilin et al, “Liposome-Polymer Systems. Introduction of Liposomes into a Polymer Gel and Preparation of the Polymer Gel inside a Liposome”, in Polymer. Sci. U.S.S.R., 30:2307-2312 (1988).
US 20160184222 A1

1.-81. (canceled)

82. An injectable formulation comprising:
a hydrogel comprising one or more liposome compositions, and
a therapeutic agent,
wherein the therapeutic agent is present within the liposome composition, and wherein the therapeutic agent is additionally present within the hydrogel but outside of the liposome composition.

83. The injectable formulation of claim 82, wherein the hydrogel comprises a first liposome composition, a second liposome composition, and a third liposome composition.

84. The injectable formulation of claim 83, wherein the first liposome composition, the second liposome composition, and the third liposome composition comprise a combination of DMPC, DOPE, DSPG, and cholesterol.

85. The injectable formulation of claim 83, wherein the first liposome composition comprises a combination of DMPC, DOPE, DSPG, and cholesterol at a ratio of 60:10:0:30, wherein the second liposome composition comprises a combination of DMPC, DOPE, DSPG, and cholesterol at a ratio of 65:5:5:25, and wherein the third liposome composition comprises a combination of DMPC, DOPE, DSPG, and cholesterol at a ratio of 60:5:5:30.

86. The injectable formulation of claim 83, wherein the hydrogel further comprises a fourth liposome composition, a fifth liposome composition, and a sixth liposome composition.

87. The injectable formulation of claim 86, wherein the fourth liposome composition, the fifth liposome composition, and the sixth liposome composition each comprise a PEG moiety on its surface.

88. The injectable formulation of claim 87, wherein the fourth liposome composition, the fifth liposome composition, and the sixth liposome composition comprise a combination of DMPC, DOPE, DSPG, and cholesterol.

89. The injectable formulation of claim 88, wherein the fourth liposome composition comprises a combination of DMPC, DOPE, DSPG, and cholesterol at a ratio of 60:10:0:30, wherein the fifth liposome composition comprises a combination of DMPC, DOPE, DSPG, and cholesterol at a ratio of 65:5:5:25, and wherein the sixth liposome composition comprises a combination of DMPC, DOPE, DSPG, and cholesterol at a ratio of 60:5:5:30.

90. The injectable formulation of claim 82, wherein the therapeutic agent is a retinal therapeutic agent.

91. The injectable formulation of claim 82, wherein the therapeutic agent is an anti-cancer therapeutic agent.

92. The injectable formulation of claim 82, wherein the therapeutic agent is an anti-Parkinson's disease therapeutic agent.

93. The injectable formulation of claim 82, wherein the therapeutic agent is a therapeutic agent for treating age related macular degeneration.

94. The injectable formulation of claim 82, wherein the therapeutic agent is an anti-VEGF antibody.

95. The injectable formulation of claim 82, wherein the therapeutic agent is bevacizumab.

96. The injectable formulation of claim 82, wherein the therapeutic agent is L-DOPA or an L-DOPA metabolite.

97. The injectable formulation of claim 82, wherein the therapeutic agent is selected from the group consisting of methadone, bupropion, buprenorphine, naloxone, naltrexone, or a combination thereof.

98. A biodegradable implant comprising an expandable hollow matrix comprising pores and the injectable formulation of claim 82,
wherein the expandable hollow matrix has a proximal end and a distal end,
wherein the expandable hollow matrix has an interior defined by a wall of the expandable hollow matrix and extending between the proximal end to the distal end thereof, and
wherein the expandable hollow implant has an unexpanded configuration and an expanded configuration.

99. The biodegradable implant of claim 98, wherein the biodegradable implant comprises a thermoplastic biodegradable polymer.

100. The biodegradable implant of claim 98, wherein the thermoplastic biodegradable polymer is selected from the group consisting of polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, polyamino acids, polymethyl vinyl ether, and copolymers, terpolymers, and any combination thereof selected from the group consisting of polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, polyamino acids, polymethyl vinyl ether, and copolymers, terpolymers, and any combination thereof.

101. A biodegradable implant comprising an expandable hollow matrix comprising pores and the injectable formulation of claim 89,
wherein the expandable hollow matrix has a proximal end and a distal end,
wherein the expandable hollow matrix has an interior defined by a wall of the expandable hollow matrix and extending between the proximal end to the distal end thereof, and
wherein the expandable hollow implant has an unexpanded configuration and an expanded configuration.
